(19) United States
(12) Patent Application Publication
(10) Pub. No.: US 2026/0115220 A1
(43) Pub. Date: Apr. 30, 2026

(54) TREATMENT AND/OR PREVENTION OF SKELETAL MUSCLE ATROPHY BY INHIBITION OF EDA2R-NIK AND OSM-OSMR SIGNALING PATHWAYS

(71) Applicant: KOC UNIVERSITESI, Istanbul (TR)

(72) Inventor: Serkan KIR, Istanbul (TR)

(73) Assignee: KOC UNIVERSITESI, Istanbul (TR)

(21) Appl. No.: 19/026,497

(22) Filed: Jan. 17, 2025

Related U.S. Application Data
(63) Continuation-in-part of application No. 18/994,067.

(30) Foreign Application Priority Data
Jan. 17, 2024 (TR) 2024/000548

(51) Int. Cl. A61K 31/7105; A61K 45/06; A61P 21/00; C07K 16/00

(52) U.S. Cl. CPC A61K 31/7105; A61K 45/06; A61P 21/00; C07K 16/00

(57) ABSTRACT

A method of using an EDA2R inhibitor or an NIK inhibitor and an OSMR inhibitor for the treatment and/or prevention of the relevant disease is provided, where the treatment and/or prevention is performed by therapeutic targeting of newly discovered mechanisms causing skeletal muscle atrophy. The present invention also proposes methods for developing, preparing, and applying pharmaceutical formulations including the relevant inhibitors.

### CROSS REFERENCE TO THE RELATED APPLICATIONS

This application is a continuation-in-part application of International Application No. PCT/TR2024/050055, filed on Jan. 24, 2024, which is based upon and claims priority to Turkish Patent Application No. 2024/000548, filed on Jan. 17, 2024, the entire contents of which are incorporated herein by reference.

### TECHNICAL FIELD

The present invention discloses a method of using inhibitors mentioned in the description for the treatment and/or prevention of the relevant disease by therapeutic targeting of newly discovered mechanisms causing skeletal muscle atrophy. The present invention also proposes methods for developing, preparing, and applying pharmaceutical formulations including the relevant inhibitors.

### BACKGROUND

Skeletal muscle atrophy is characterized by reduced muscle mass and strength and often leads to physical dysfunction and reduced quality of life [1]. Muscle atrophy process involves an imbalance between protein synthesis and breakdown, with the latter being predominant. This imbalance leads to a net loss in muscle proteins, causing muscle fibers and overall muscle mass to shrink. In other words, skeletal muscle atrophy is characterized by excessive protein catabolism which leads to loss of muscle mass and strength [2]. There is no effective treatment against muscle wasting because the mechanisms driving the atrophy process are not completely understood.

The loss of skeletal muscle tissue is a serious health problem, which is associated with multiple disease states that are diverse in pathology. Progressive muscle loss is a serious comorbidity associated with many chronic diseases (such as cancer, kidney disease, heart failure, obstructive lung disease, diabetes, acquired immunodeficiency syndrome (AIDS), and Cushing's syndrome which occurs due to excessive cortisol production through adrenal glands). Rapid muscle loss also manifests in acute diseases such as burn trauma and sepsis. In the most severe cases, muscle loss may evolve into critical illness myopathy, in which the failure of respiratory skeletal muscles can lead to death. Moreover, muscle loss may develop as a physiological response to fasting, malnutrition, and aging. Aging-associated muscle loss is known as sarcopenia and affects more than half of individuals over the age of 80. In addition, patients with genetic muscular dystrophies, such as Duchenne muscular dystrophy (DMD), Facioscapulohumeral (FSHD) and Limb-girdle (LGMD) muscular dystrophies, suffer from muscle loss due to inherent defects in the myofibril structure. Amyotrophic lateral sclerosis (ALS), which is a neurodegenerative disease, involves the denervation of muscles and leads to muscle atrophy. Furthermore, muscle loss can be localized to specific muscles in individuals who suffer spinal cord injuries or joint immobilization due to plaster casts.

Muscle atrophy, which remains unresolved, is a negative prognostic factor for the progression of the underlying disease. This is particularly evident in cancer-associated cachexia, which is an atrophy syndrome involving the loss of muscle and adipose tissues. Muscle loss can also be associated with aging (i.e., sarcopenia), muscular dystrophies, and cachexia syndrome which is linked to chronic diseases such as cancer and kidney failure. Cachexia involves progressive muscle wasting, which is often accompanied by the loss of adipose tissue. Cachexia is a multifactorial syndrome characterized by systemic inflammation, fatigue, anorexia, and other behavioral changes [3]. Almost half of all cancer patients suffer from cachexia, which is responsible for at least 20% of all cancer deaths. Cachexia is highly prevalent in patients with lung, gastric, pancreatic, or colorectal cancers and leads to dramatic weight loss and poor quality of life. The frequency of weight loss in patients with pancreatic and gastric cancer is over 60-80%, while the incidence of weight loss in patients with lung, colorectal, prostate or head and neck cancer is over 50%. In these patients, muscle loss negatively affects survival by reducing tolerance of treatment and interfering with the response to treatment. Muscle loss also causes frailty and poor quality of life due to restrained physical activity. Although the prevalence of muscle loss in adults is very high, there is currently no effective treatment to reverse this complication. Treatment and/or prevention of muscle atrophy is critical for the survival of patients and their quality of life [4]. A better understanding of tumor-driven mechanisms which promote muscle atrophy is needed to design novel therapeutics.

A variety of signaling pathways are involved in the regulation of muscle mass and function. These include the tumor necrosis factor (TNF) family cytokine, the interleukin-6 (IL-6) family cytokine, and the myostatin/activin pathway. These pathways can affect muscle mass and function by modulating protein synthesis and breakdown, inflammation, and other cellular processes.

Ectodysplasin A (EDA) is a Tumor Necrosis Factor (TNF) family member involved in ectodermal development. Mutations in the EDA gene have been associated with X-linked hypohidrotic ectodermal dysplasia, which is a congenital disease characterized by abnormalities in the development of skin, hair, nails, teeth, and sweat glands [5]. Alternative splicing generates numerous EDA transcripts, including well-known isoforms EDA-A1 and EDA-A2, which differ only by two amino acids missing in EDA-A2. While EDA-A1 binds to the EDAR receptor, EDA-A2 only interacts with ectodysplasin a2 receptor (EDA2R) [6]. These ligands are produced as transmembrane proteins, but they are also enzymatically cleaved and secreted. Defects in EDA and EDAR genes are linked to ectodermal dysplasia [7]. EDA2R, which is a member of the TNF receptor family, is known to be involved in the development of ectodermal tissues, but its function in skeletal muscle is not yet clearly understood. [8].

Another pathway, which also attracts attention, includes Oncostatin M (OSM) and Oncostatin M receptor (OSMR). OSM, which is a member of IL-6 cytokine family, is involved in a variety of biological processes, including inflammation and tissue remodeling.

Despite the progress made for understanding the molecular mechanisms underlying muscle atrophy, effective treatments to prevent or reverse this condition are still lacking. Current therapeutic strategies, such as the use of TNFα inhibitors, anti-IL-6 antibodies, and myostatin inhibitors have shown limited success. These treatments may improve muscle mass in some patients, but they often fail to improve muscle function and physical performance.

US20180021407A1, known in the state of the art, discloses the use of polypeptides or agents described in the said document for the treatment of diseases such as cancer, by binding to TNF receptor superfamily proteins and inducing and/or enhancing the immune response.

In the scientific and medical communities, there is a continuous attempt to better understand the molecular mechanisms causing muscle atrophy and to identify novel therapeutic targets, which is the subject of the present invention, in the literature. No technique has been found which conducts studies on inhibiting EDA2R-NIK and OSM-OSMR signaling pathways for the treatment and/or prevention of skeletal muscle atrophy.

### SUMMARY

The present invention relates to the use of at least one EDA2R inhibitor or NFκB-inducing kinase (NIK) inhibitor in the treatment and/or prevention of a musculoskeletal disease, in particular skeletal muscle atrophy disease.

The present invention aims to ameliorate loss of muscle mass and function by therapeutic targeting of newly discovered mechanisms causing skeletal muscle atrophy.

In the said invention, EDA2R-NIK and OSM-OSMR pathways are identified as triggers of muscle atrophy, and it is shown that their blockage prevents muscle loss. Thus, it is aimed to improve the quality of life and survival of patients affected by this condition.

The objective of the present invention relates to the use of EDA2R inhibitor or NIK inhibitor in the treatment and/or prevention of skeletal muscle atrophy.

In the present invention, EDA2R-NIK and OSM-OSMR pathways are identified as drivers of muscle atrophy. The elements of these pathways, including EDA-A2, EDA2R, NIK, OSM, and OSMR, have not been targeted to prevent loss of muscle mass and function in any other prior studies and their related function has not previously been detected. Previously reported studies have targeted TNFα, IL6-, myostatin/activin, ghrelin, and androgen receptor pathways and there has been no report on preventing significant levels of loss of muscle mass and function.

EDA2R inhibitor or NIK inhibitor, which is the active substance used in the present invention, prevents tumor-induced muscle loss by acting on OSM-OSMR and EDA2-EDA2R pathways. It has shown a protective/therapeutic effect in skeletal muscle atrophy and no side effects have been observed. By means of the present invention, potential therapeutic effects of the inhibitor for use in the treatment of skeletal muscle atrophy have been demonstrated.

In addition, the EDA2R inhibitor or NIK inhibitor can be used alone or in combination with at least one OSMR inhibitor.

Another objective of the present invention relates to realizing and preparing a pharmaceutical formulation including the above-mentioned inhibitor as active substance in the said invention.

A further objective of the present invention relates to developing pharmaceutical formulations including therapeutically effective amount of at least one pharmaceutically acceptable EDA2R inhibitor or NIK inhibitor and at least one pharmaceutically acceptable excipient and the use thereof as a drug in the treatment of skeletal muscle atrophy.

The present invention also relates to a method for the treatment of patients (subjects) affected by skeletal muscle atrophy disease, and includes the following steps:
a) selecting a patient who is found to be affected by skeletal muscle atrophy disease,
b) administering a therapeutically effective amount of EDA2R inhibitor or NIK inhibitor to the patient.

### BRIEF DESCRIPTION OF THE DRAWINGS

“Treatment and/or Prevention of Skeletal Muscle Atrophy by Inhibition of EDA2R-NIK and OSM-OSMR Signaling Pathways”, which is realized to achieve the objective of the present invention is illustrated in the accompanying figures, in which:

FIGS. 1A-1M. EDA-A2 promotes atrophy and activates NFκB signaling in myotubes. FIGS. 1A-1B, Eda2r mRNA was tested by RT-qPCR in gastrocnemius muscles of mice bearing LLC tumors (16 days) (control n=5, LLC n=7) or B16 tumors (14 days) (n=5) (FIG. 1A) and in quadriceps muscle biopsies of lung and colorectal cancer patients with or without cachexia (w/o cachexia n=16, with cachexia n=18) (FIG. 1B). FIGS. 1C-1D, EDA2R transcript was analyzed in rectus abdominis muscle biopsies collected from non-cancer controls (n=15) and PDAC patients with cachexia (n=17) and without cachexia (n=5) (GSE130563) (FIG. 1C) and in quadriceps muscle biopsies collected from non-cancer subjects (n=6) and cachectic patients with upper gastrointestinal cancer (n=12) before and after tumor resection (GSE34111) (FIG. 1D). FIG. 1E, Mouse primary myotubes were treated with recombinant EDA-A2 (250 ng/mL; 24 hr.) and gene expression was determined by RT-qPCR (n=3). FIGS. 1F-1G, Mouse primary myotubes transduced with a GFP adenovirus were treated with recombinant EDA-A1, EDA-A2 or TNFα proteins (250 ng/mL each; 48 hr.) and visualized under the fluorescence microscope. Average myotube diameter was measured (n=3) (FIG. 1F). The scale bar is 50 μm (FIG. 1G). FIGS. 1H-1I, Human Skeletal Muscle Myoblasts differentiated into myotubes were treated with an adenovirus expressing EDA-A2 (FIG. 1H) or recombinant EDA-A2 protein (250 ng/mL; 48 hr.) (FIG. 1I). Gene expression was determined by RT-qPCR (n=3) (FIG. 1H) and myotube diameter was measured (n=3) (FIG. 1I). FIGS. 1J-1M, Mouse primary myotubes were treated with EDA-A1, EDA-A2 or TNFα (250 ng/mL each) for 24 hours (FIG. 1J, FIG. 1L, and FIG. 1M) or 10 minutes (FIG. 1K). Gene expression was determined by RT-qPCR (n=3) (FIG. 1J). Cell lysates were investigated by western blotting (FIG. 1K and FIG. 1L) and treated with alkaline phosphatase (FIG. 1M). The values are mean±SEM. Each dot represents a biological replicate. The data represent two (FIG. 1H, FIG. 1I, and FIG. 1J) or three (FIG. 1A, FIG. 1E, FIG. 1F, FIG. 1G, FIG. 1K, FIG. 1L, and FIG. 1M) independent experiments. Statistical analysis was conducted by using the two-tailed unpaired t-test (FIG. 1A, FIG. 1B, FIG. 1C, FIG. 1E, FIG. 1H, and FIG. 1I) and one-way ANOVA with Tukey's multiple-comparison test (FIG. 1D, FIG. 1F, and FIG. 1J).

FIGS. 2A-2G. Activation of the noncanonical NFκB signaling by EDA-A2, or NIK kinase induces atrophy in primary myotubes. FIG. 2A, Mouse primary myotubes were treated with IKKβ inhibitor TPCA-1 (10 μM), proteasome inhibitor MG132 (5 μM), and EDA-A2 (250 ng/mL) for 24 hours. Changes in gene expression were determined by RT-qPCR (n=3). FIGS. 2B-2C, Primary myotubes were treated with TPCA-1 (10 μM) or proteasome inhibitor MG132 (5 μM), and EDA-A2 (250 ng/mL) for 10 minutes (FIG. 2B) or 24 hours (FIG. 2C) and protein levels of NFκB signaling components were examined by western blotting. FIGS. 2D-2E, Primary myotubes were treated with recombinant EDA-A2, BAFF or TWEAK proteins (250 ng/mL each) for 24 hours. Protein levels were determined by western blotting (FIG. 2D) and gene expression was examined by RT-qPCR (n=3) (FIG. 2E). FIGS. 2F-2G, Primary myotubes were transduced with adenoviruses expressing LacZ or human NIK (hNIK). After 24 hours, changes in gene expression were determined by RT-qPCR (n=3) (FIG. 2F). Myotubes, which were also treated with GFP adenovirus, were visualized under the fluorescence microscope after 48 hours. The scale bar is 50 μm (FIG. 2G). The values are mean±SEM. Each dot represents a biological replicate. The data represent two (FIGS. 2D-2E) or three (FIG. 2A, FIG. 2B, FIG. 2C, FIG. 2F, and FIG. 2G) independent experiments. Statistical analysis was conducted by using one-way ANOVA with Tukey's multiple-comparison test (FIG. 2A and FIG. 2E) and the two-tailed unpaired t-test (FIG. 2F).

FIGS. 3A-3I. EDA-A2-induced muscle atrophy requires NIK kinase activity. FIGS. 3A-3B, Mouse primary myotubes were treated with NIK kinase inhibitor B022 (5 μM) and EDA-A2 (250 ng/mL) for 24 hours. Changes in gene expression were tested by RT-qPCR (n=3) (FIG. 3A) and protein levels were determined by western blotting (FIG. 3B). FIGS. 3C-3D, Primary myotubes transduced with GFP adenovirus were treated with B022 (5 μM) and EDA-A2 (250 ng/mL) for 48 hours. Cells were visualized under the fluorescence microscope and myotube diameters were measured (n=3). The scale bar is 50 μm. FIGS. 3E-3I, Tibialis anterior muscles of mice were transduced with LacZ or EDA-A2 adenoviruses. Mice were sacrificed after 7 days (n=5 mice per group). Changes in gene expression were determined by RT-qPCR (n=5 mice) (FIG. 3E). Tissues were weighed (FIG. 3F) and H&E stained (FIG. 3G). Muscle fiber cross-sectional area (CSA) (FIG. 3H) and the fiber frequency distribution were determined (FIG. 3I) (n=3 mice). The scale bar is 100 μm. The values are mean±SEM. Each dot represents a biological replicate. The data represent two (FIG. 3B, FIG. 3G, FIG. 3H, and FIG. 3I) or three (FIG. 3A, FIG. 3C, FIG. 3D, FIG. 3E, and FIG. 3F) independent experiments. Statistical analysis was conducted by using one-way ANOVA with Tukey's multiple-comparison test (FIG. 3A and FIG. 3C) and the two-tailed unpaired t-test (FIG. 3E, FIG. 3F, and FIG. 3H).

FIGS. 4A-4I. EDA2R-deficient mice are resistant to tumor-induced muscle loss. FIGS. 4A-4B and FIGS. 4E-4I, Mice were inoculated with LLC cells and sacrificed after 16 days. Collected tissues were weighed (n=6 mice per group) (FIG. 4A). Forelimb grip strength was measured before the sacrifice (EDA2R-KO-LLC n=5, other groups n=6 mice) (FIG. 4B). FIGS. 4C-4D, Mice were inoculated with B16 cells and sacrificed after 14 days (EDA2R-KO-B16 n=5, other groups n=6 mice). Forelimb grip strength was measured before the sacrifice (FIG. 4C). Collected tissues were weighed (FIG. 4D). FIGS. 4E-4G, Gastrocnemius muscle cross-sections were H&E stained (FIG. 4E), cross-sectional area (FIG. 4F) and the fiber frequency distribution (FIG. 4G) were measured (n=6 mice). The scale bar is 100 μm. FIG. 4H and FIG. 4I, mRNA levels of gastrocnemius muscle were tested by RT-qPCR (n=6 mice) (FIG. 4H) and protein levels were determined by western blotting (n=3 mice) (FIG. 4I). The values are mean±SEM. Each dot represents a biological replicate. The data represent three independent experiments (FIGS. 4A-4I). Statistical analysis was conducted by using two-way ANOVA with Tukey's multiple-comparison test (FIG. 4A, FIG. 4B, FIG. 4C, FIG. 4D, FIG. 4F, and FIG. 4H).

FIGS. 5A-5L. OSM induces Eda2r expression in muscle and the depletion of OSMR protects from muscle loss. FIG. 5A, Mouse primary myotubes were treated with recombinant OSM (250 ng/mL) for 6 hours (n=3). FIG. 5B, Tibialis anterior muscles of mice were transduced with LacZ or OSM adenoviruses. Mice were sacrificed after 7 days. mRNA levels were determined by RT-qPCR (n=6 mice). FIG. 5C, Mice were inoculated with LLC cells and sacrificed after 16 days. Plasma OSM levels were measured by ELISA (control n=8, LLC n=7 mice). FIG. 5D, Mouse primary myotubes were treated with recombinant OSM (250 ng/mL for 48 hr) and EDA-A2 (100 ng/mL for 24 hr.). Changes in gene expression were determined by RT-qPCR (n=3). FIG. 5E, OSMR levels in gastrocnemius muscle were determined by immunohistochemistry. FIGS. 5F-5L, Mice were inoculated with LLC cells and sacrificed after 16 days (WT-LLC n=5, other groups n=6 mice). Collected tissues were weighed (FIG. 5F). Forelimb grip strength was measured before the sacrifice (WT n=6, other groups n=5 mice) (FIG. 5G). Gastrocnemius muscle cross-sections were H&E stained (FIG. 5I), cross-sectional area (FIG. 5H) and the fiber frequency distribution (FIG. 5J) were measured (WT-LLC n=5, other groups n=6 mice). The scale bar is 100 μm. FIG. 5K and FIG. 5L, mRNA levels of gastrocnemius muscle were tested by RT-qPCR (WT-LLC n=5, other groups n=6 mice) (FIG. 5K) and protein levels were determined by western blotting (n=3 mice) (FIG. 5L). The values are mean±SEM. Each dot represents a biological replicate. The data represent two (FIG. 5A, FIG. 5C, FIG. 5D, and FIG. 5E) or three (FIG. 5B, FIGS. 5F-5L) independent experiments. Statistical analysis was conducted by using the two-tailed unpaired t-test (FIGS. 5A-5C), one-way ANOVA with Tukey's multiple-comparison test (FIG. 5D), and two-way ANOVA with Tukey's multiple-comparison test (FIG. 5F, FIG. 5G, FIG. 5H, and FIG. 5K).

FIGS. 6A-6F. OSM promotes cellular atrophy in cultured primary myotubes.

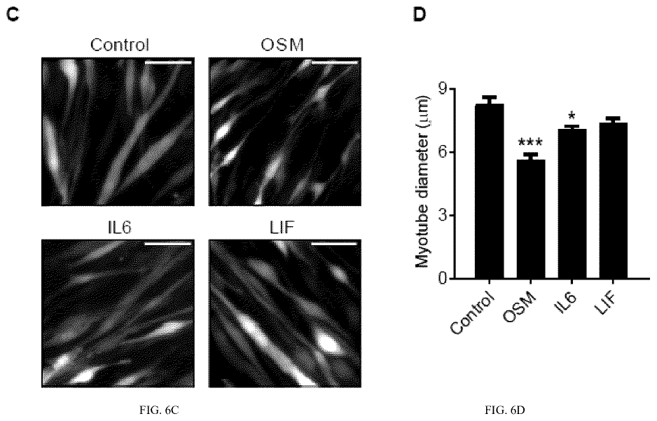

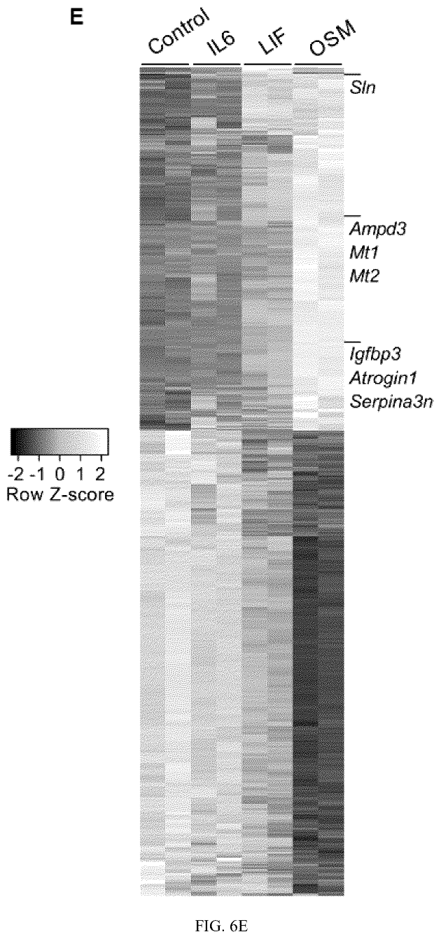

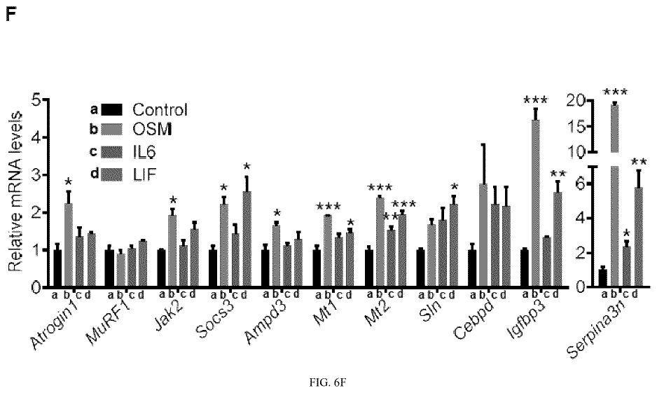

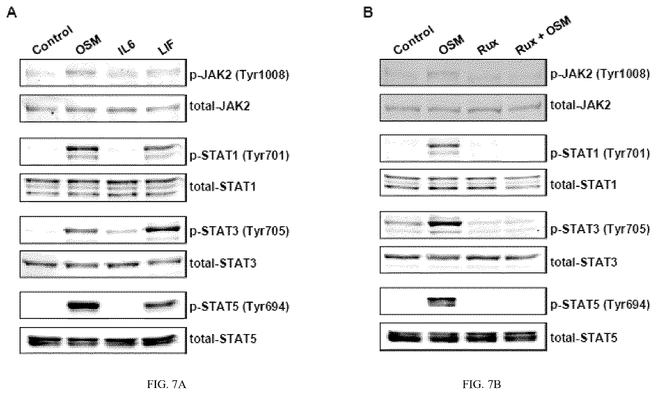

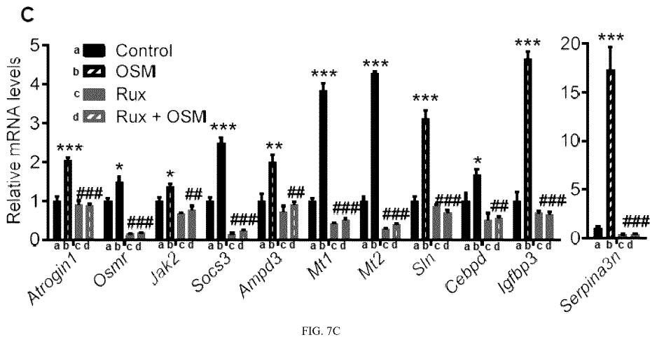

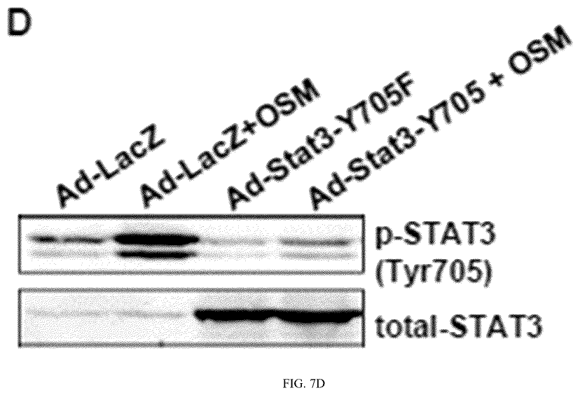

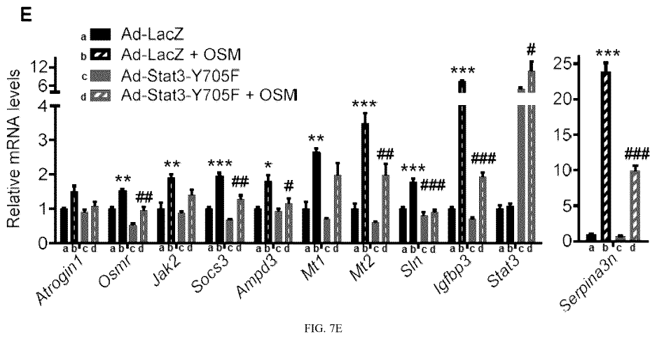

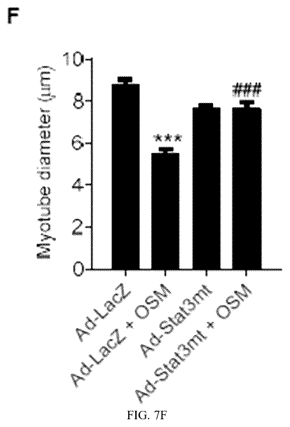

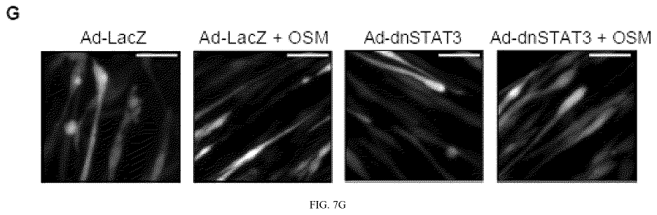

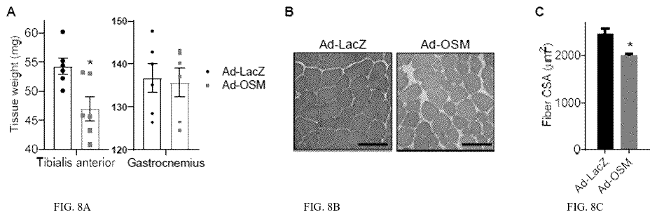

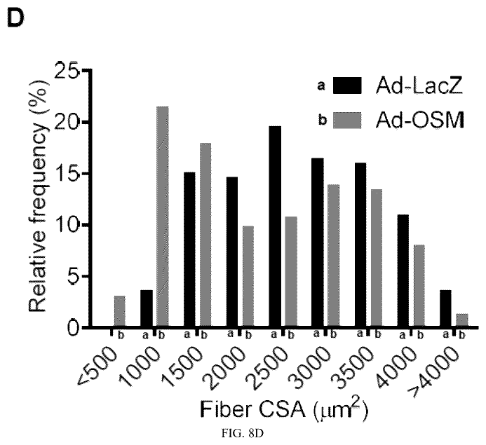

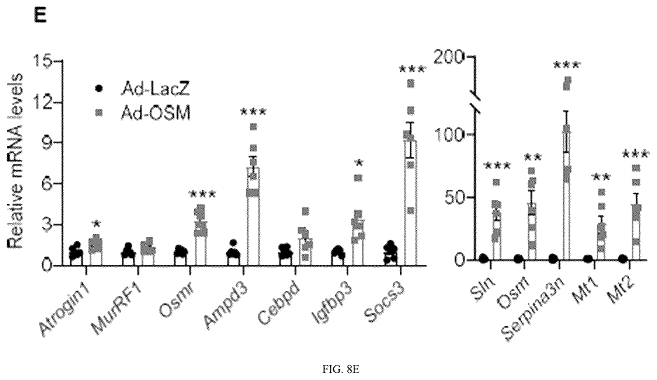

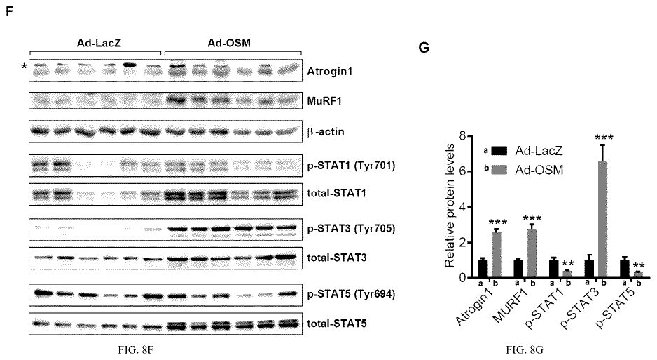

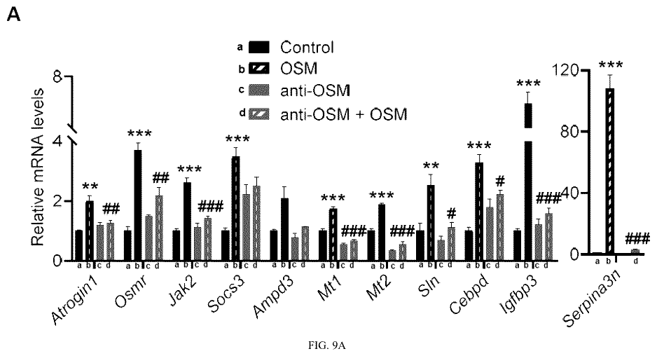

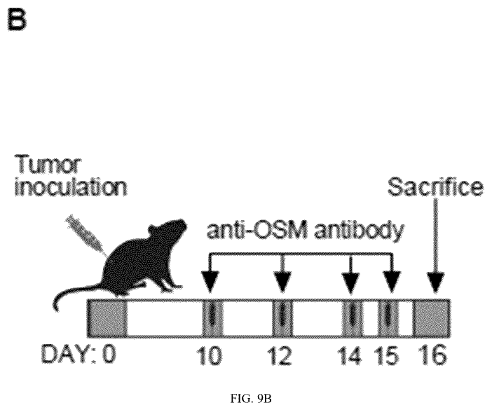

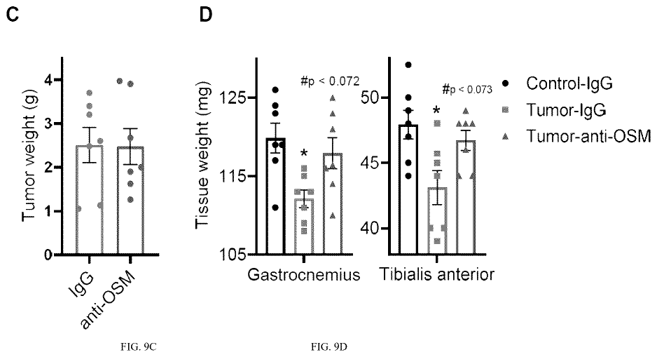

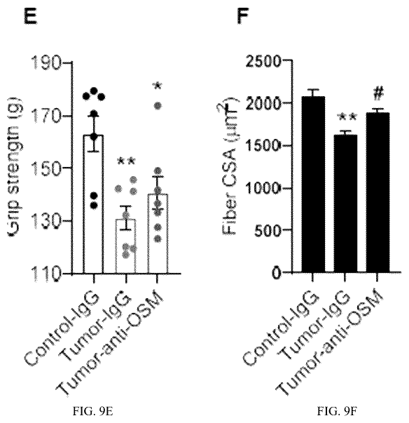

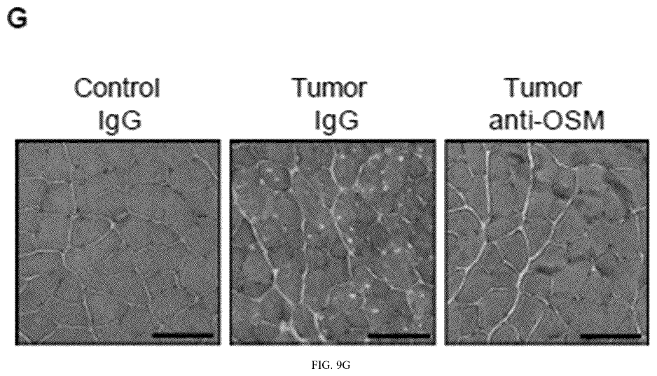

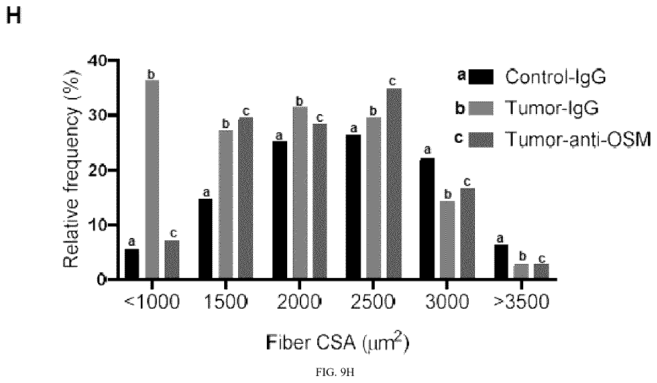

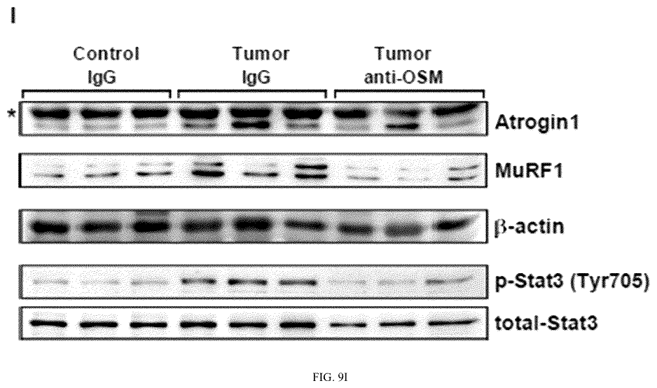

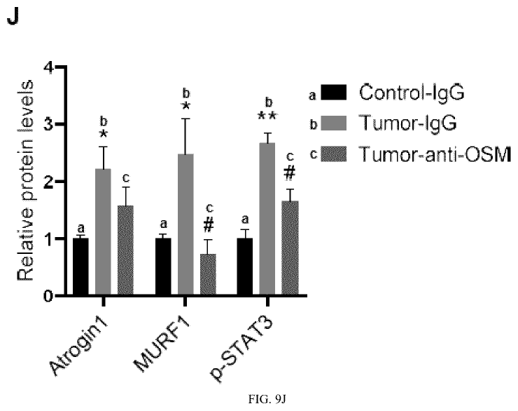

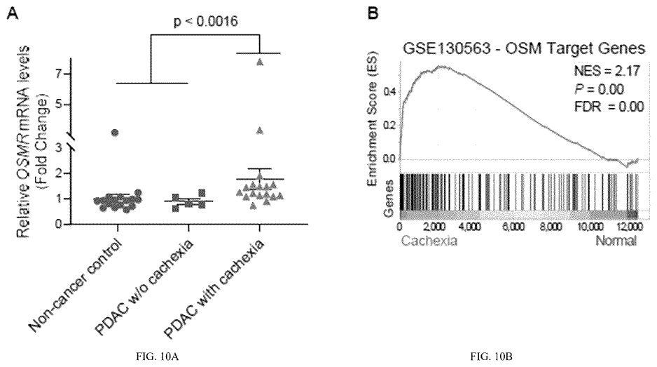

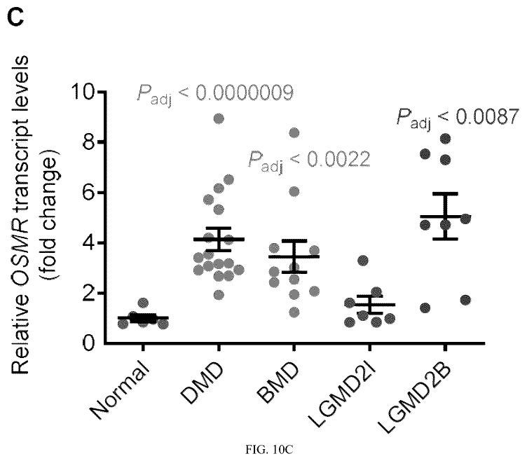

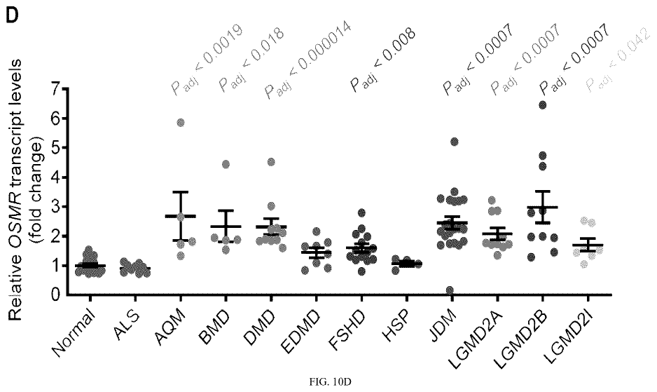

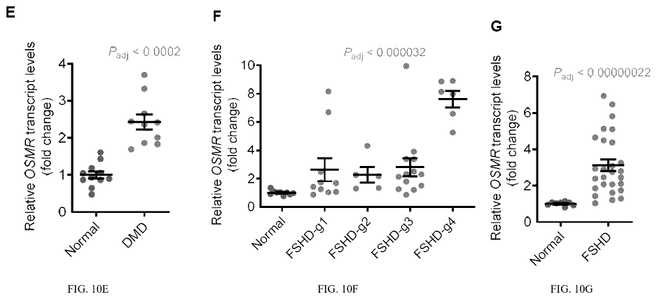

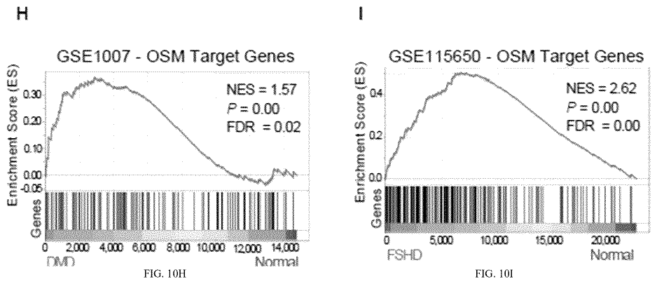

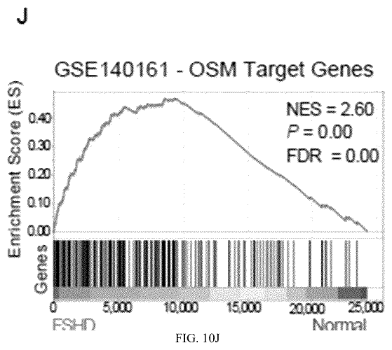

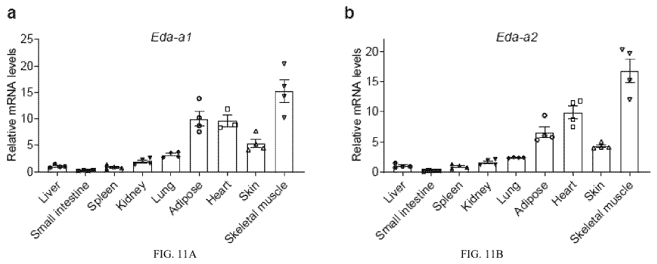

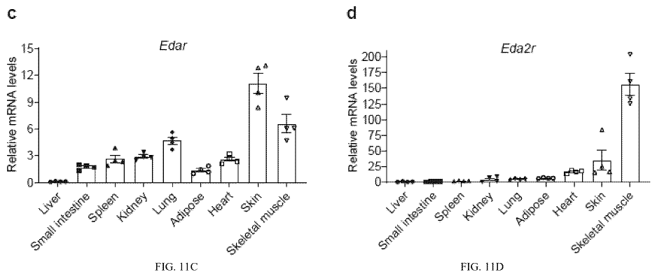

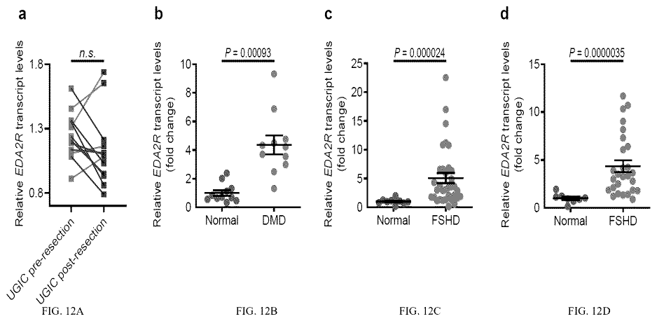

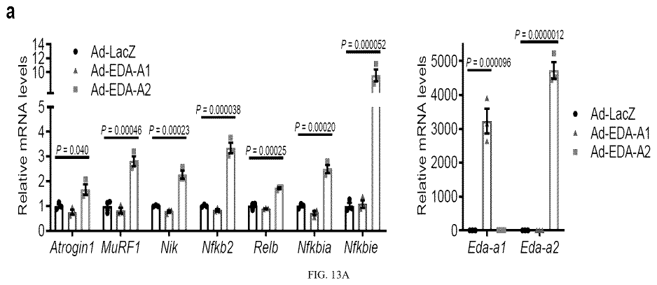

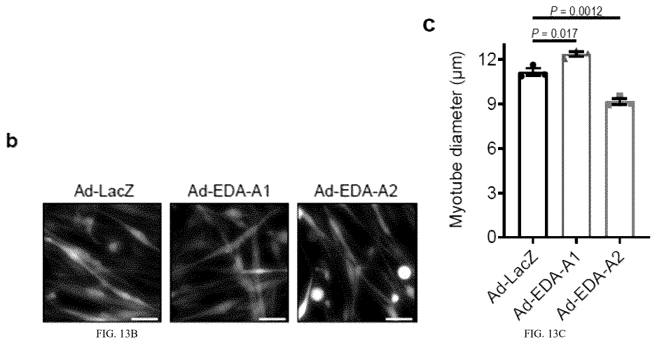

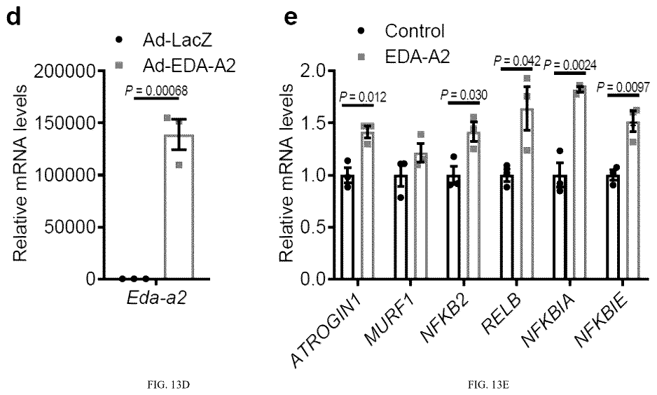

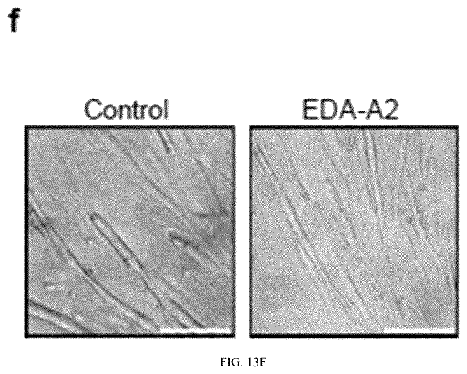

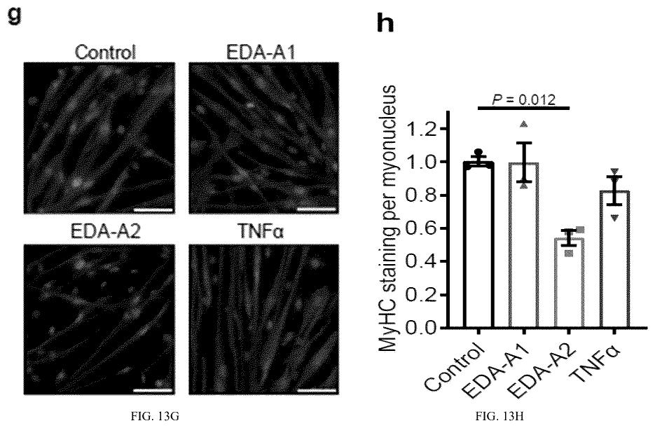

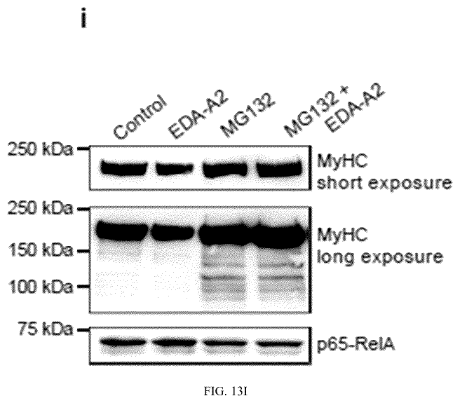

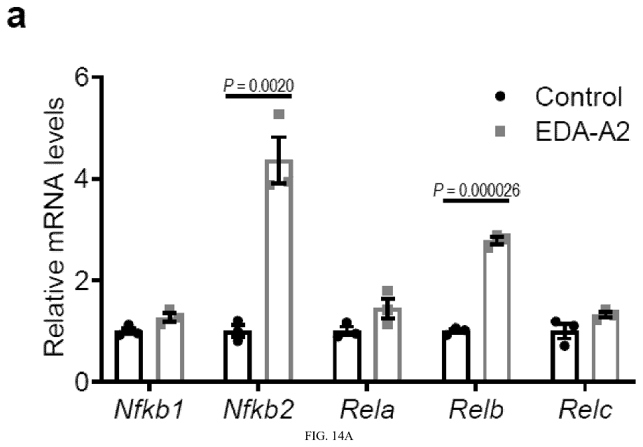

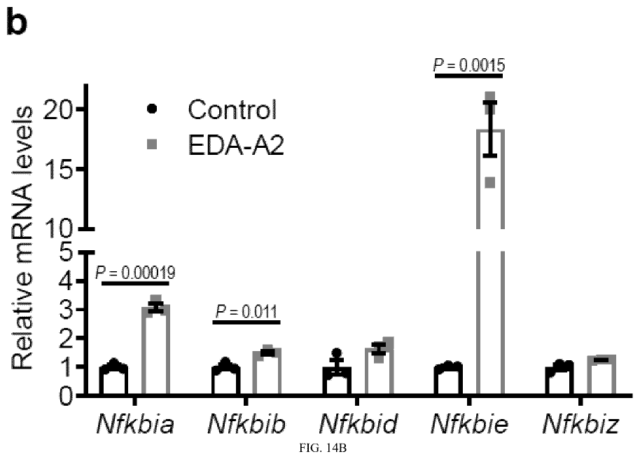

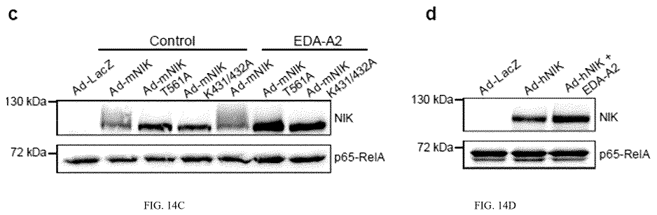

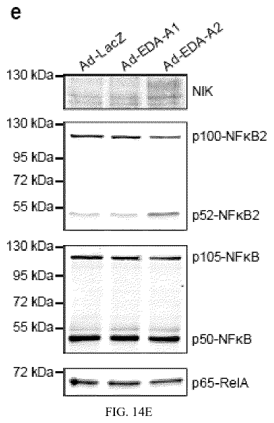

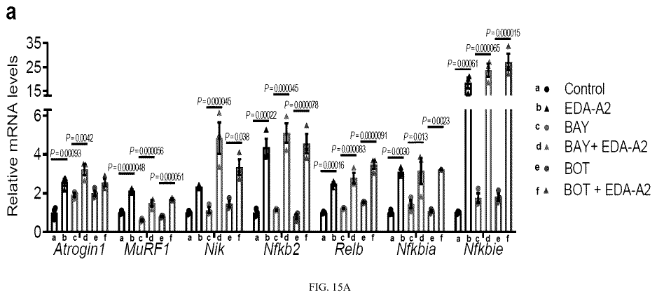

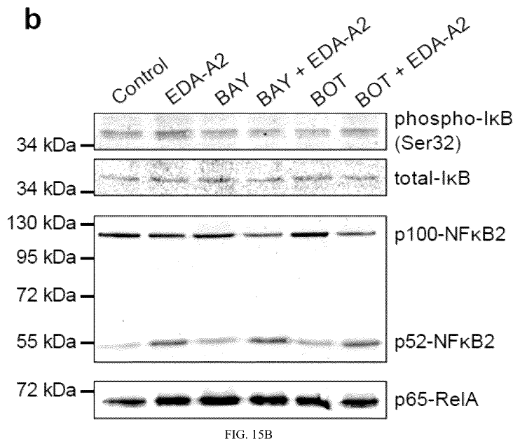

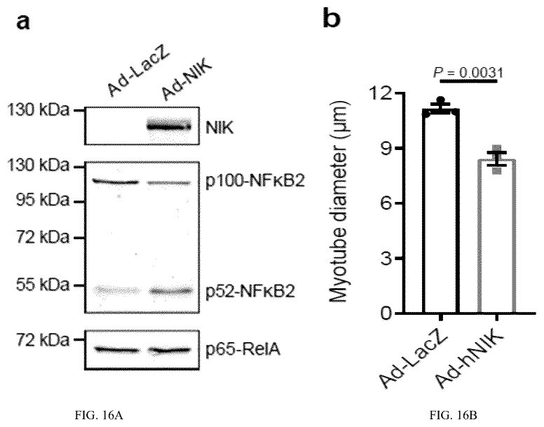

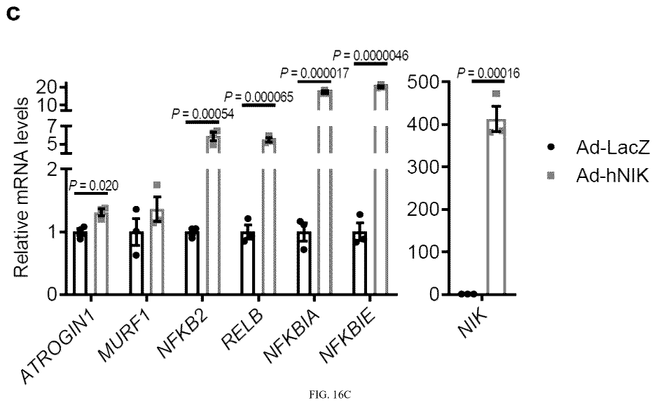

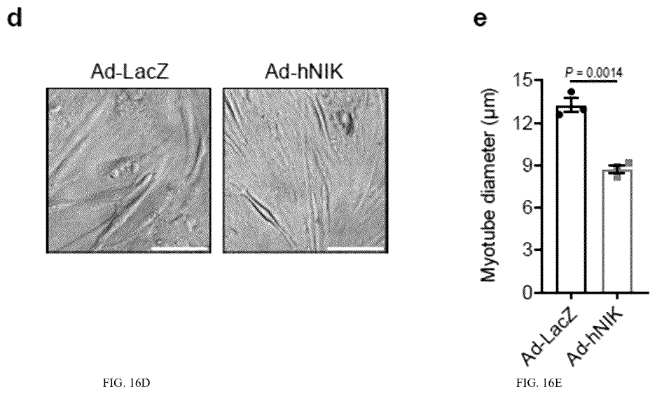

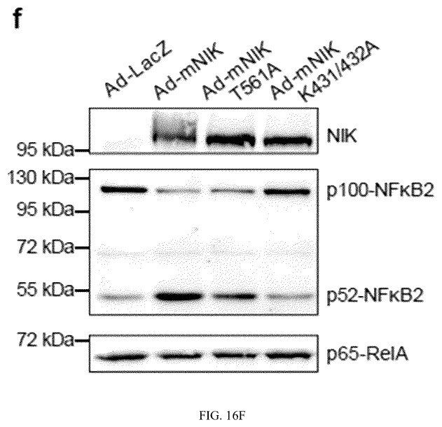

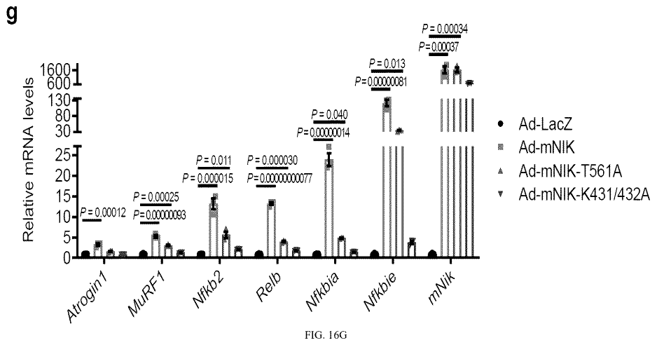

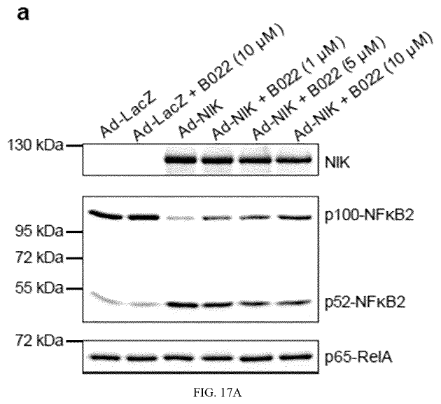

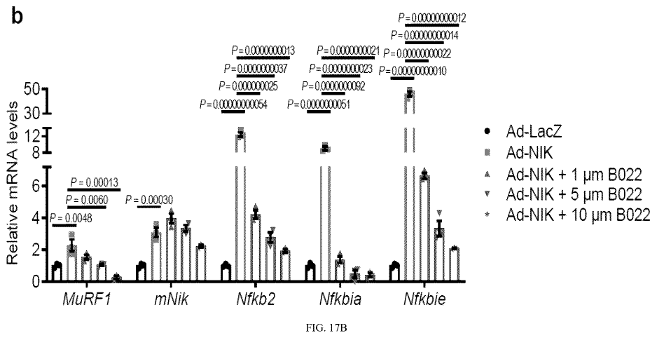

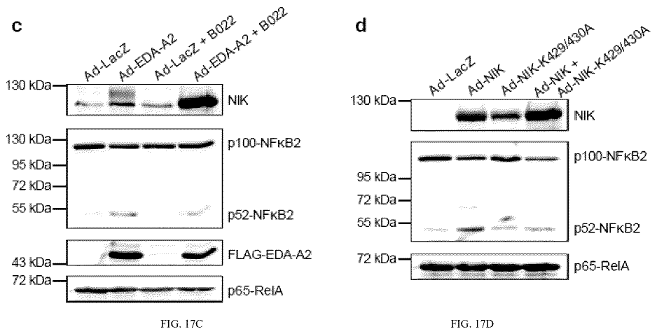

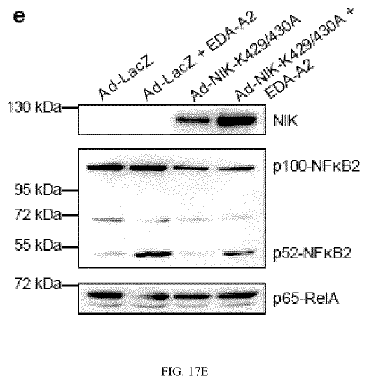

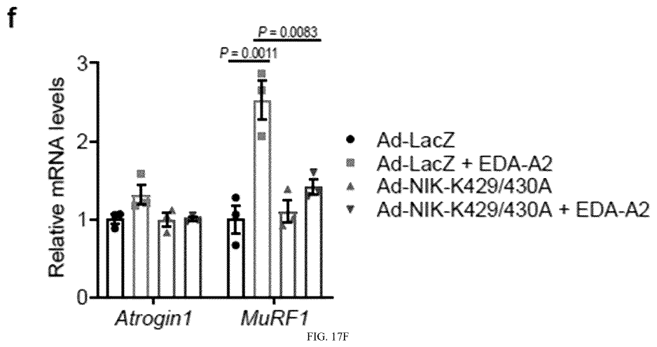

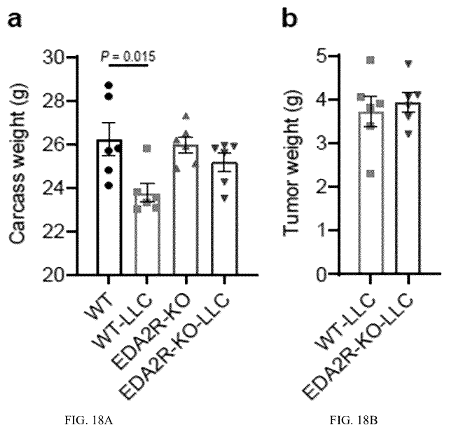

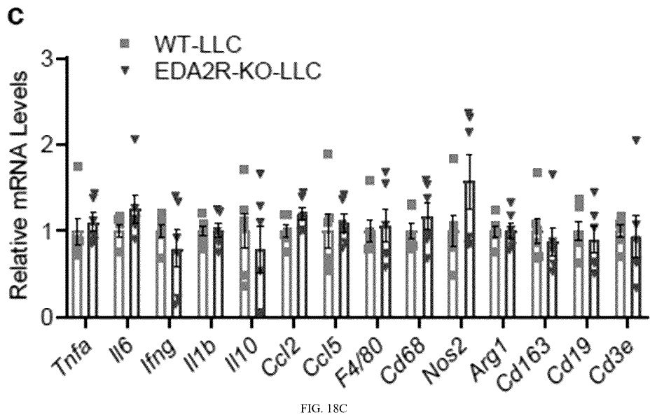

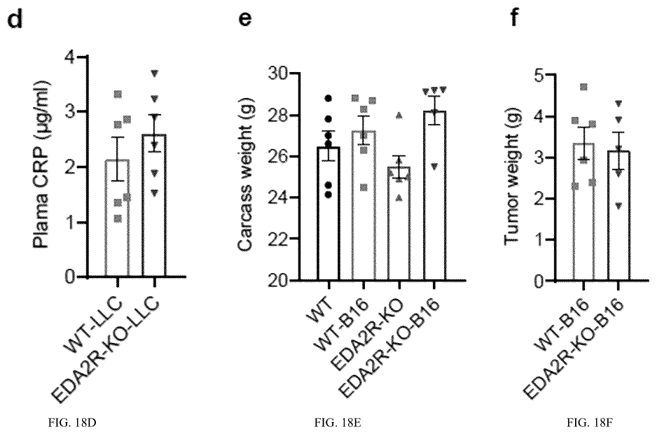

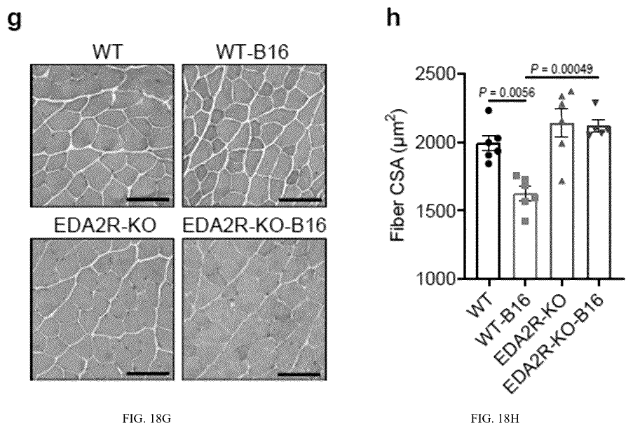

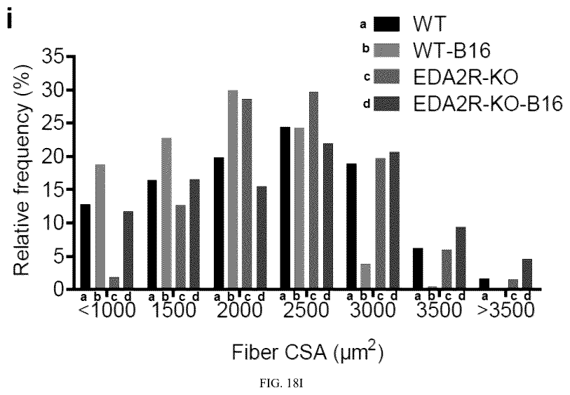

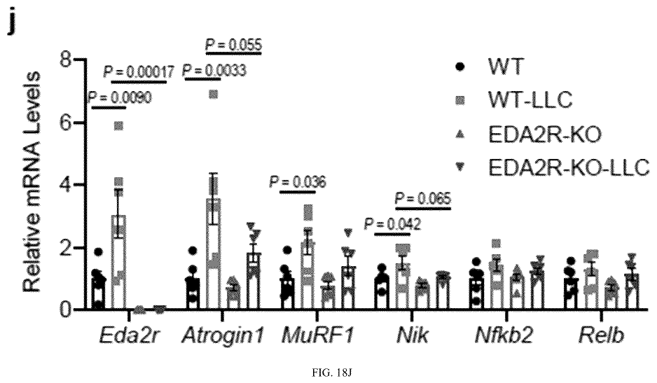

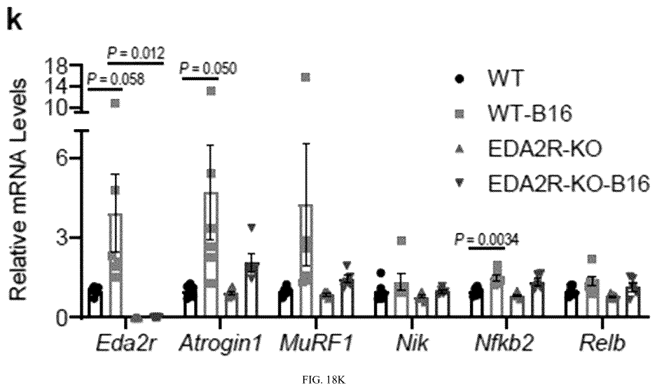

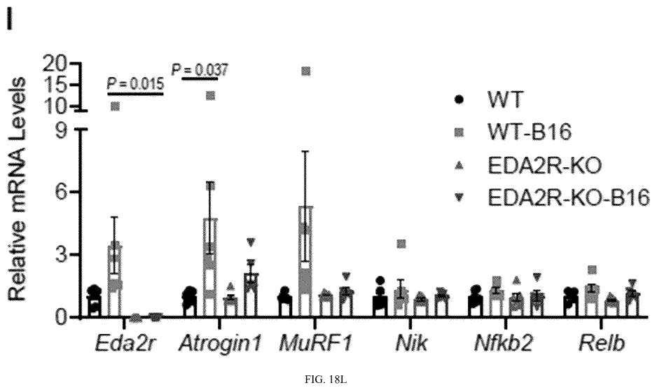

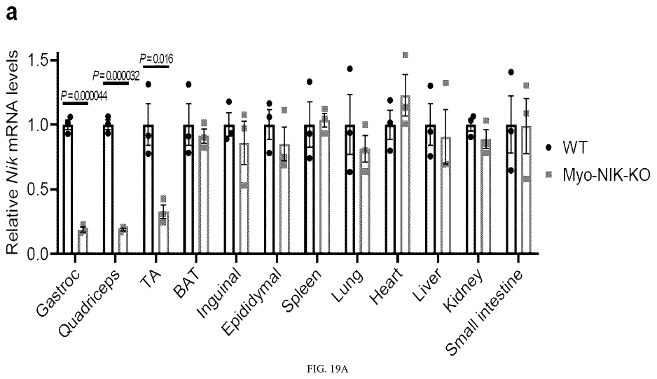

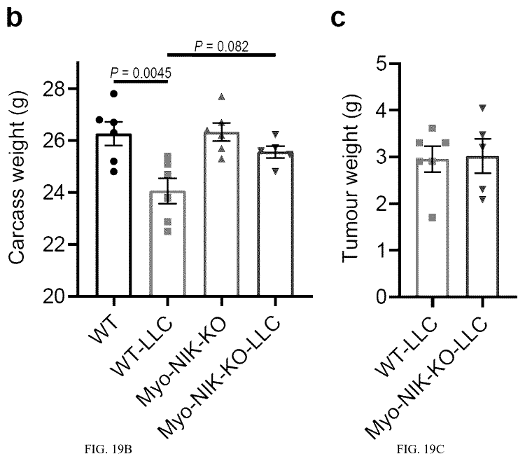

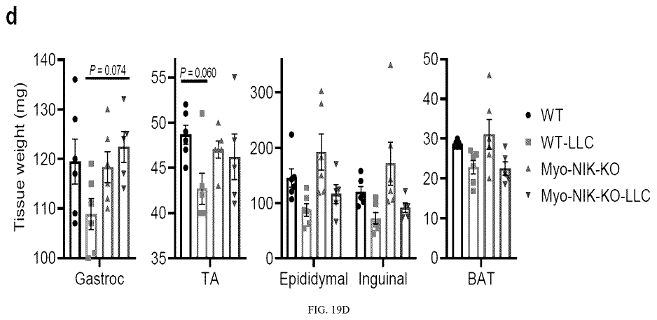

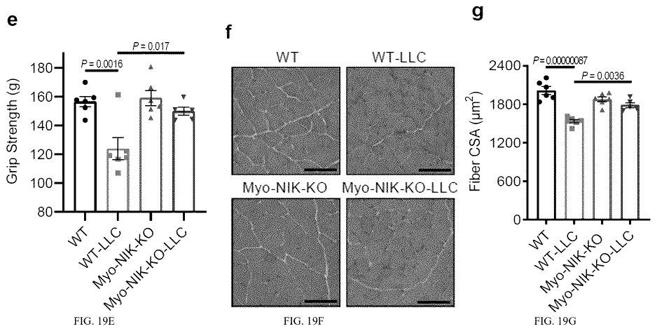

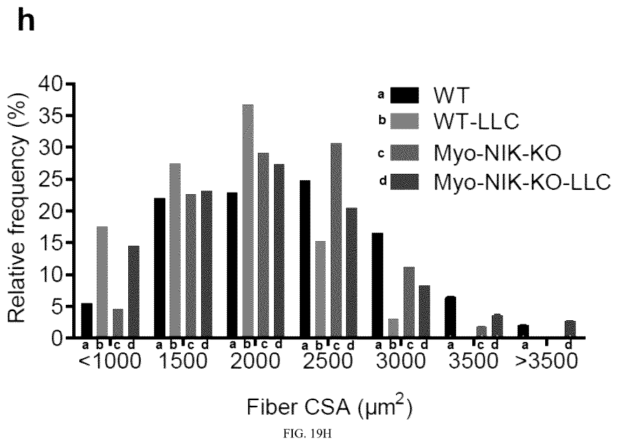

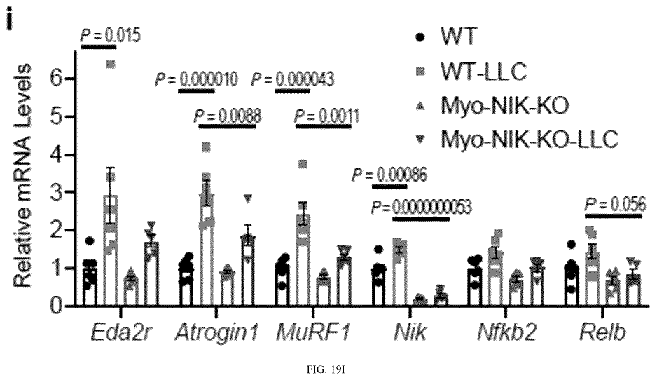

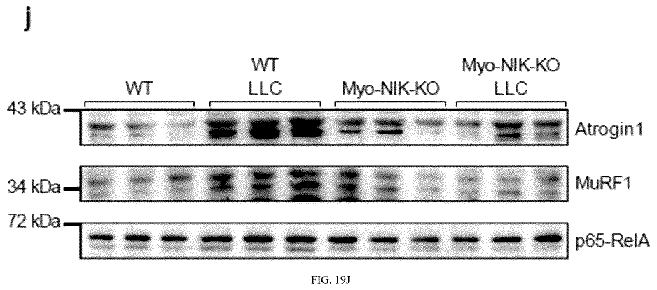

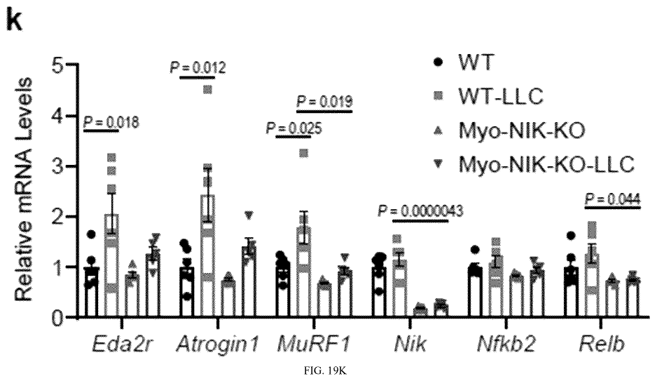

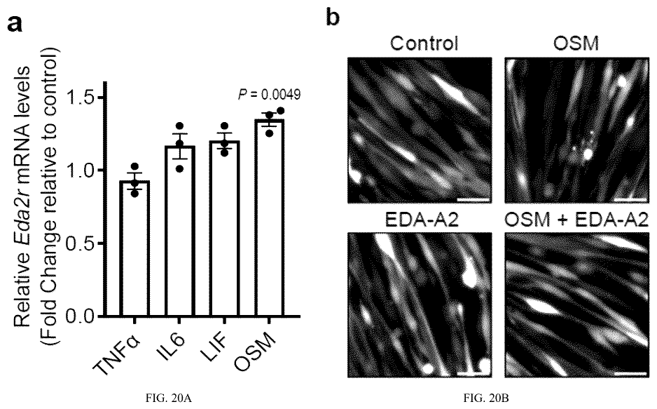

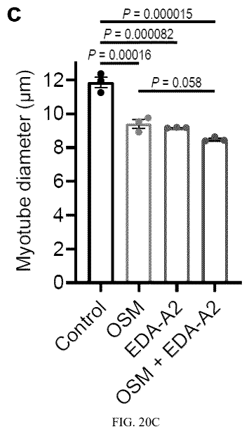

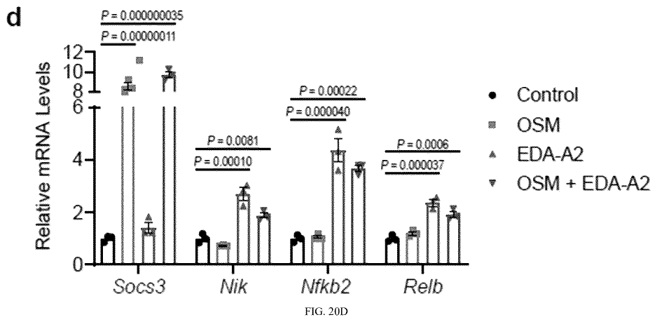

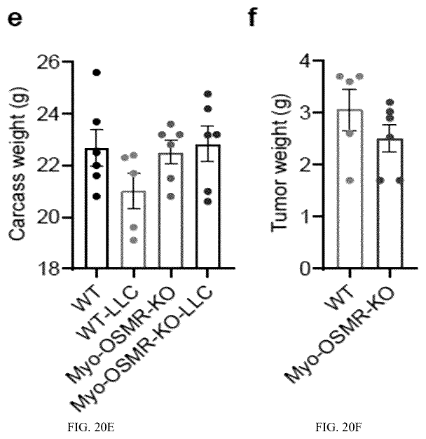

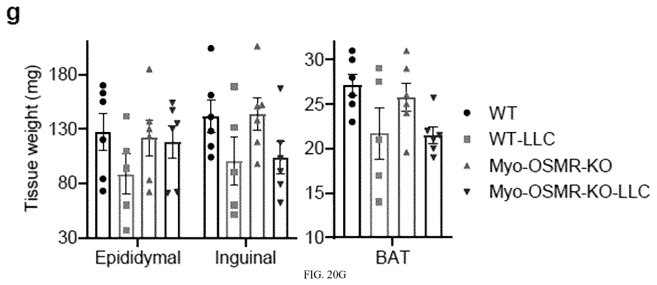

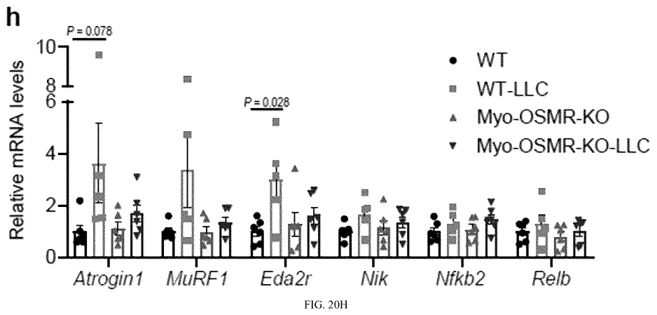

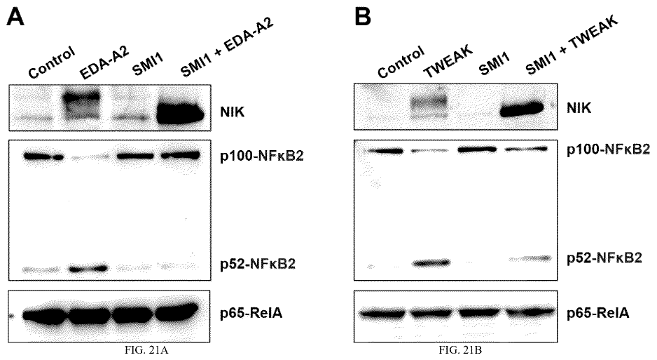

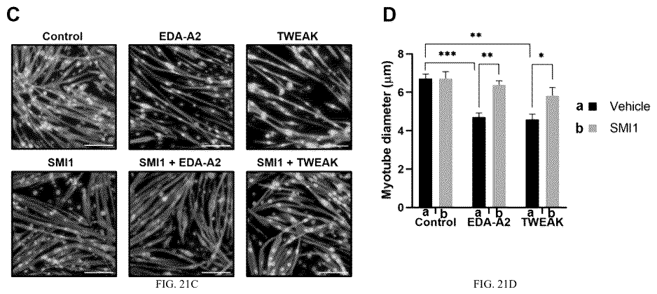

(FIG. 6A) C57BL/6 mice inoculated with LLC cells were sacrificed after 16 days and changes in gene expression of gastrocnemius muscle were determined by RT-qPCR (n=5 for the control group and n=7 for the tumor group). (FIG. 6B) Mouse primary myotubes were treated with recombinant OSM (250 ng/mL) for 48 hours. Changes in gene expression were determined by RT-qPCR (n=3 for each group). (FIGS. 6C-6D) Mouse primary myotubes were transduced with a GFP adenovirus. Cells were treated with recombinant OSM, IL6 or LIF (each 250 ng/mL) for 48 hours and then visualized under the fluorescence microscope. Scale bar is 50 μm (FIG. 6C). Average myotube diameter was measured (n=4 for each group) (FIG. 6D). (FIGS. 6E-6F) Mouse primary myotubes were treated with recombinant OSM, IL6 or LIF (each 250 ng/mL) for 48 hours. Gene expression profiles were analyzed by RNA sequencing. Heatmap of significant genes, which were up-or down-regulated more than two-fold, is shown (n=2 for each group) (FIG. 6E). Changes in gene expression were determined by RT-qPCR (n=3 for each group) (FIG. 6F). Data are presented as mean±SEM. Statistical analysis was conducted by using two-tailed t-test (FIGS. 6A-6B) and one-way ANOVA with Tukey's post-hoc test (FIG. 6D and FIG. 6F). *p<0.05, **p<0.01, ***p<0.001, when compared with the control group.

FIGS. 7A-7G. OSM utilizes JAK/STAT3 signaling to elicit its effects in myotubes.

(FIG. 7A) Mouse primary myotubes were treated with recombinant OSM, IL6 or LIF (each 250 ng/mL) for 10 minutes. Protein levels were determined by western blotting.

(FIG. 7B) Mouse primary myotubes were treated with Ruxolitinib (Rux; 2 μM) for 30 minutes and then recombinant OSM (250 ng/mL) was added for 10 minutes. Protein levels were determined by western blotting.

(FIG. 7C) Mouse primary myotubes were treated with Ruxolitinib (Rux; 2 μM) and recombinant OSM (250 ng/mL) for 48 hours. mRNA levels were determined by RT-qPCR (n=3 for each group). *p<0.05, **p<0.01, ***p<0.001 compares the differences between Control and OSM groups.

##p<0.01, ###p<0.001 compares the differences between OSM and Rux+OSM groups.

(FIGS. 7D-7G) Mouse primary myotubes were transduced with LacZ or Stat3-Y705F expressing adenoviruses and treated with recombinant OSM (250 ng/mL) for 48 hours. Protein levels were determined by western blotting.

(FIG. 7D). mRNA levels were tested by RT-qPCR (n=3 for each group) (FIG. 7E). Cells were also transduced with a GFP adenovirus for fluorescence imaging. Average myotube diameter was measured (n=4 for each group) (FIG. 7F). Myotubes were visualized under the fluorescence microscope. Scale bar is 50 μm (FIG. 7G). *p<0.05, **p<0.01, ***p<0.001 compares the differences between Ad-LacZ and Ad-LacZ+OSM groups. #p<0.05, ##p<0.01, ###p<0.001 compares the differences between Ad-LacZ+OSM and Ad-Stat3-Y705F+OSM groups.

Data are presented as mean±SEM. Statistical analysis was conducted by using one-way ANOVA with Tukey's post-hoc test.

FIGS. 8A-8G. Overexpression of OSM causes muscle atrophy in mice.

(FIGS. 8A-8G) Tibialis anterior muscles of C57BL/6 mice were transduced with LacZ or OSM expressing adenoviruses. Mice were sacrificed after 7 days (n=6 for each group). Tissues were weighed (FIG. 8A) and H&E stained (FIG. 8B). The scale bar is 100 μm. Muscle fiber cross-sectional area (CSA) (FIG. 8C) and the fiber frequency distribution were determined (FIG. 8D) (n=3 for each group). Changes in gene expression were determined by RT-qPCR (n=6 for each group) (FIG. 8E). Protein levels were tested by western blotting. Asterisk (*) indicates non-specific band (FIG. 8F). Band intensities were quantified (FIG. 8G) (n=6 for each group). Data are presented as mean±SEM. Statistical analysis was conducted by using two-tailed t-test. *p<0.05, *p<0.01, ***p<0.001, when compared with the Ad-LacZ group.

FIGS. 9A-9J. Neutralization of OSM ameliorates tumor-induced muscle loss.

(FIG. 9A) Mouse primary myotubes were treated with recombinant OSM (250 ng/mL) and IgG or anti-OSM antibodies (10 μg/mL) for 48 hours. Changes in gene expression were determined by RT-qPCR (n=3 for each group).
*p<0.01, ***p<0.001 compares the differences between IgG and IgG+OSM groups. #p<0.05, ##p<0.01, ###p<0.001 compares the differences between IgG+OSM and anti-OSM+OSM groups.

(FIGS. 9B-9H) Mice inoculated with LLC cells received IgG or anti-OSM antibody injections and were sacrificed 16 days after tumor inoculation. Tumor (FIG. 9C) and muscle tissues (FIG. 9D) were weighed. Forelimb grip strength was measured before the sacrifice (n=7 for each group) (FIG. 9E). Gastrocnemius muscle cross-sections were H&E stained (FIG. 9G), cross-sectional area (CSA) (FIG. 9F) and the fiber frequency distribution (FIG. 9H) were measured (n=3 for each group). The scale bar is 100 μm. Protein levels of gastrocnemius muscle were determined by western blotting. Asterisk (*) indicates non-specific band (FIG. 9I). Band intensities were quantified (FIG. 9J) (n=3 for each group). *p<0.05, **p<0.01, compares the differences between Control-IgG and Tumor-IgG groups. #p<0.05, compares the differences between Tumor-IgG and Tumor-anti-OSM groups. Data are presented as individual measurements (points) and mean±SEM. Statistical analysis was conducted by using one-way ANOVA with Tukey's post-hoc test.

FIGS. 10A-10J. OSM target genes are upregulated in muscles of cancer and muscular dystrophy patients.

(FIGS. 10A-10B) OSMR expression values of normal subjects and PDAC patients were analyzed by GEO2R (GSE130563) (n=16 for non-cancer controls, n=5 for non-cachectic PDAC patients, and n=17 for cachectic PDAC patients) (FIG. 10A). Gene set enrichment analysis (GSEA) of the top 200 OSM target genes was performed by comparing cachectic PDAC patients with non-cancer controls and non-cachectic PDAC patients (GSE130563) (FIG. 10B).

(FIG. 10C) OSMR expression values of normal subjects and DMD patients were analyzed by GEO2R (GSE109178) (n=6 for normal subjects, n=17 for DMD subjects, n=11 for Becker muscular dystrophy (BMD) subjects, n=7 for fukutin-related protein (FKRP)-deficient limb girdle muscular dystrophy type 2I (LGMD2I) subjects and n=8 for dysferlin-deficient limb-girdle muscular dystrophy type 2B (LGMD2B) subjects).

(FIG. 10D) OSMR expression values of normal subjects and muscular dystrophy patients were analyzed by GEO2R (GSE3307) (n=18 for normal subjects, n=9 for amyotrophic lateral sclerosis (ALS) patients, n=5 for Acute quadriplegic myopathy (AQM), n=5 for Becker muscular dystrophy (BMD) patients, n=10 for DMD patients, n=8 for Emery Dreifuss muscular dystrophy (EDMD) patients, n=14 for FSHD patients, n=4 for hereditary spastic paraplegia (HSP) patients, n=21 for juvenile dermatomyositis (JDM) patients, n=10 for calpain3-deficient limb girdle muscular dystrophy type 2A (LGMD2A) patients, n=10 for dysferlin-deficient limb girdle muscular dystrophy type 2B (LGMD2B) patients, and n=7 for fukutin-related protein (FKRP)-deficient limb girdle muscular dystrophy type 2I (LGMD2I) patients).

(FIG. 10E) OSMR expression values of normal subjects and DMD patients were analyzed by GEO2R (GSE1007) (n=11 for normal subjects and n=10 for DMD subjects).

(FIGS. 10F-10G) OSMR expression values of normal subjects and FSHD patients were analyzed by DESeq2 (GSE115650) (n=9 for normal subjects and n=34 for FSHD subjects) (FIG. 10F), (GSE140261) (n=8 for normal subjects and n=27 for FSHD subjects) (FIG. 10G).

(FIGS. 10H-10J) Gene set enrichment analysis (GSEA) of the top 200 OSM target genes was performed. Enrichment plots for DMD (GSE1007) (FIG. 10H), and FSHD (GSE115650) (FIG. 10I), (GSE140261) (FIG. 10J) compared to respective normal subjects are shown.

Data are presented as individual points and mean±SEM. Statistical analysis was conducted by using GEO2R or DESeq2 and values were adjusted for multiple tests.

FIGS. 11A-11D. The expression of Eda-a2 and Eda2r is enriched in skeletal muscle tissue. FIGS. 11A-11D, Various tissue samples were collected from C57BL/6 mice. Relative mRNA levels were determined by RT-qPCR (n=4 mice). The values are mean±SEM. Each dot represents a biological replicate. The data represent two independent experiments (FIGS. 11A-11D).

FIGS. 12A-12D. EDA2R expression is induced in DMD and FSHD patients. FIG. 12A, EDA2R transcript levels were analyzed in quadriceps muscle biopsies collected from cachectic patients with upper gastrointestinal cancer (n=12) before and after tumor resection (GSE34111). Upon surgery, EDA2R levels were upregulated in 4 patients (red connecting lines) and downregulated in 8 patients (green connecting lines). n.s. statistically not significant. FIG. 12B, GSE1007 dataset was analyzed by GEO2R and EDA2R expression values were determined in normal individuals and DMD patients (n=10). In each group, one individual has 2 technical replicates and the total number of data points is 11. FIG. 12C, GSE115650 dataset was analyzed by DESeq2 and EDA2R expression values were determined in normal individuals (n=9) and FSHD patients (n=34). FIG. 12D, GSE140261 dataset was analyzed by DESeq2 and EDA2R expression values were determined in normal individuals (n=8) and FSHD patients (n=27). The values are mean±SEM. Statistical analysis was conducted by using the two-tailed paired t-test (FIG. 12A). Adjusted P values were calculated with Benjamini & Hochberg false discovery rate method by GEO2R (FIGS. 12B-12C) and DESeq2 (FIG. 12D).

FIGS. 13A-13I. The overexpression of EDA-A2 or the administration of recombinant EDA-A2 in human and mouse myotubes stimulates cellular atrophy. FIGS. 13A-13C, Mouse primary myotubes were transduced with LacZ, EDA-A1, or EDA-A2 expressing adenoviruses. After 24 hours, gene expression was tested by RT-qPCR (n=3) (FIG. 13A). Myotubes, which were also treated with GFP adenovirus, were visualized under the fluorescence microscope after 48 hours. The scale bar is 50 μm (FIG. 13B). Average myotube diameter was measured (n=3) (FIG. 13C). FIGS. 13D-13F, Human Skeletal Muscle Myoblasts (HSMM) were differentiated into myotubes and treated with an adenovirus expressing EDA-A2 (FIG. 13D) or recombinant EDA-A2 protein (250 ng/mL) (FIGS. 13E-13F) for 48 hours. Gene expression was determined by RT-qPCR (n=3) (FIGS. 13D-13E). Human myotubes were examined under the light microscope. The scale bar is 100 μm (FIG. 13F). g, h, Mouse primary myotubes were treated with recombinant EDA-A1, EDA-A2 or TNFα proteins (250 ng/mL each) for 48 hours. MyHC was immunofluorescently labeled, while nuclei were counterstained with DAPI. The scale bar is 50 μm (FIG. 13G). MyHC signal was normalized to the number of myotube nuclei (n=3) (FIG. 13H). FIG. 13I, Mouse primary myotubes, which were treated with recombinant EDA-A2 (250 ng/mL) and proteasome inhibitor MG132 (10 μM), were lysed, and protein samples were examined by western blotting. The values are mean±SEM. Each dot represents a biological replicate. The data represent two (FIGS. 13A-13D and FIGS. 13F-13I) or three (FIG. 13E) independent experiments. Statistical analysis was conducted by using one-way ANOVA with Tukey's multiple-comparison test (FIG. 13A, FIG. 13C, and FIG. 13H) or the two-tailed unpaired t-test (FIGS. 13D-13E).

FIGS. 14A-14E. EDA-A2 stimulates the expression of NFκB signaling components and the alternative NFκB activation in primary myotubes. Electrophoretic mobility shift of mouse NIK protein depends on its autophosphorylation and kinase activity. FIGS. 14A-14B, Mouse primary myotubes were treated with recombinant EDA-A2 (250 ng/mL) for 24 hours and gene expression was determined by RT-qPCR (n=3). FIGS. 14C-14D, Mouse primary myotubes were transduced with adenoviruses expressing LacZ, wild-type mouse NIK (mNIK), autophosphorylation-deficient mNIK-T561A mutant, and kinase-dead mNIK-K431/432A mutant or human NIK (hNIK). A day later, recombinant EDA-A2 (250 ng/mL) was also added for another 24 hours. Protein levels were determined by western blotting. FIG. 14E, Mouse primary myotubes were transduced with LacZ, EDA-A1, or EDA-A2 expressing adenoviruses. After 24 hours, protein levels were determined by western blotting. The values are mean±SEM. Each dot represents a biological replicate. The data represent two (FIGS. 14B-14C and FIG. 14E) or three (FIGS. 14A-14B) independent experiments. Statistical analysis was conducted by using the two-tailed unpaired t-test.

FIGS. 15A-15B. Activation of the canonical NFκB signaling is not required for EDA-A2-induced gene expression in primary myotubes. FIGS. 15A-15B, Mouse primary myotubes were treated with IκB phosphorylation inhibitors BAY 11-7082 (10 μM) and BOT-64 (10 μM) in combination with recombinant EDA-A2 (250 ng/mL) for 24 hours. Gene expression was studied by RT-qPCR (n=3) (FIG. 15A) and protein levels were determined by western blotting (FIG. 15B). The values are mean±SEM. Each dot represents a biological replicate. The data represent two independent experiments. Statistical analysis was conducted by using one-way ANOVA with Tukey's multiple-comparison test.

FIGS. 16A-16G. Overexpression of NIK promotes the alternative NFκB activation and atrophy in primary myotubes. FIGS. 16A-16B, Mouse primary myotubes were transduced with adenoviruses expressing LacZ or human NIK (hNIK). After 24 hours, protein levels were determined by western blotting (FIG. 16A). Myotubes, which were also treated with GFP adenovirus, were visualized under the fluorescence microscope after 48 hours and average myotube diameter was measured (n=3) (FIG. 16B). FIGS. 16C-16E, Human Skeletal Muscle Myoblasts (HSMM) were differentiated into myotubes and treated with adenoviruses expressing LacZ or human hNIK for 48 hours. Gene expression was determined by RT-qPCR (n=3) (FIG. 16C). Human myotubes were examined under the light microscope. The scale bar is 100 μm (FIG. 16D). Myotube diameter was measured (n=3) (FIG. 16E). FIGS. 16F-16G, Primary myotubes were transduced with adenoviruses expressing LacZ, mouse NIK (mNIK), autophosphorylation-deficient mNIK-T561A mutant or kinase dead mNIK-K431/432A mutant. Protein levels were determined by western blotting (FIG. 16F). This is the same experiment as FIG. 14C. NIK and p65-RelA blots were cropped from FIG. 14C. mRNA levels were tested by RT-qPCR (n=3) (FIG. 16G). The values are mean±SEM. Each dot represents a biological replicate. The data represent two (FIGS. 16A-16B and FIGS. 16D-16G) or three (FIG. 16C) independent experiments. Statistical analysis was conducted by using the two-tailed unpaired t-test (FIGS. 16B-16C and FIG. 16E) and one-way ANOVA with Tukey's multiple-comparison test (FIG. 16G).

FIGS. 17A-17F. The inhibition of NIK kinase activity with B022 or a dominant-negative NIK mutant blocks the effects of EDA-A2 in primary myotubes. FIGS. 17A-17B, Mouse primary myotubes were transduced with adenoviruses expressing LacZ or human NIK and treated with different doses of B022 (1 μM, 5 μM or 10 μM) for 24 hours. Protein levels were determined by western blotting (FIG. 17A) and changes in gene expression were tested by RT-qPCR (n=3) (FIG. 17B). FIG. 17C, Mouse primary myotubes were transduced with LacZ or EDA-A2 adenoviruses and treated with B022 (5 μM) for 24 hours. Protein levels were determined by western blotting. FIG. 17D, Mouse primary myotubes were transduced with adenoviruses expressing wild-type human NIK or the dominant-negative human NIK-K429/430A mutant. Protein levels were determined by western blotting. FIGS. 17E-17F, Mouse primary myotubes were transduced with adenoviruses expressing LacZ or the NIK-K429/430A mutant and treated with recombinant EDA-A2 (100 ng/mL) for 24 hours. Protein levels were determined by western blotting (FIG. 17E) and changes in gene expression were tested by RT-qPCR (n=3) (FIG. 17F). The values are mean±SEM. Each dot represents a biological replicate. The data represent two (FIGS. 17A-17D and FIG. 17F) or three (FIG. 17E) independent experiments. Statistical analysis was conducted by using one-way ANOVA with Tukey's multiple-comparison test.

FIGS. 18A-18L. EDA2R-deficient mice are resistant to tumor-induced muscle wasting. FIGS. 18A-18D and FIG. 18J, Mice were inoculated with LLC cells and sacrificed after 16 days (n=6 mice per group). Carcass weight without the tumor mass (FIG. 18A) and tumor weight (FIG. 18B) were measured. FIG. 18C, Gene expression levels in tumor samples were measured by RT-qPCR (n=6 mice). FIG. 18D, Plasma CRP levels were determined by ELISA (n=6 mice). FIGS. 18E-18I and FIGS. 18K-18L, Mice were inoculated with B16 cells and sacrificed after 14 days (EDA2R-KO-B16 n=5, other groups n=6 mice). Carcass weight without the tumor mass (FIG. 18E) and tumor weight (FIG. 18F) were measured. LLC tumors caused a decrease in carcass weight. However, when mice were given B16 tumors, tissue loss did not affect the carcass weight. This is because these tumors mask tissue loss by causing excessive subcutaneous swelling due to inflammation. FIGS. 18G-18I, Gastrocnemius muscle cross-sections were H&E stained (FIG. 18G), cross-sectional area (FIG. 18H) and the fiber frequency distribution (FIG. 18I) were measured. The scale bar is 100 μm. FIG. 18J, mRNA levels of quadriceps muscle of the LLC tumor-bearing mice were tested by RT-qPCR (n=6 mice). FIGS. 18K-18L, mRNA levels of gastrocnemius muscle (FIG. 18K) and quadriceps muscle (FIG. 18L) of the B16 tumor-bearing mice were determined by RT-qPCR (EDA2R-KO-B16 n=5, other groups n=6 mice). The values are mean±SEM. Each dot represents a biological replicate. The data represent three independent experiments (FIGS. 18A-18L). Statistical analysis was conducted by using two-way ANOVA with Tukey's multiple-comparison test (FIG. 18A, FIG. 18H, and FIGS. 18J-18L).

FIGS. 19A-19K. Muscle-specific depletion of NIK protects from tumor-induced muscle loss. FIG. 19A. mRNA levels of Nik were tested by RT-qPCR in various tissues of the Myo-NIK-KO mice (n=3 mice). FIGS. 19B-19K, Mice were inoculated with LLC cells and sacrificed after 16 days (Myo-NIK-KO-LLC n=5, other groups n=6 mice). Carcass weight without the tumor mass (FIG. 19B) and tumor weight (FIG. 19C) were measured. Collected tissues were weighed (FIG. 19D). Forelimb grip strength was measured before the sacrifice (FIG. 19E). FIGS. 19F-19H, Gastrocnemius muscle cross-sections were H&E stained (FIG. 19F), cross-sectional area (FIG. 19G) and the fiber frequency distribution (FIG. 19H) were measured. The scale bar is 100 μm. FIGS. 19I-19J, mRNA levels of gastrocnemius muscle were tested by RT-qPCR (Myo-NIK-KO-LLC n=5, other groups n=6 mice) (FIG. 19I) and protein levels were determined by western blotting (n=3 mice) (FIG. 19J). mRNA levels of quadriceps muscle were tested by RT-qPCR (Myo-NIK-KO-LLC n=5, other groups n=6 mice) (FIG. 19K). The values are mean±SEM. Each dot represents a biological replicate. The data represent three independent experiments (FIGS. 19A-19K). Statistical analysis was conducted by using the two-tailed unpaired t-test (FIG. 19A) or two-way ANOVA with Tukey's multiple-comparison test (FIG. 19B, FIG. 19D, FIG. 19E, FIG. 19G, FIG. 19I, and FIG. 19K).

FIGS. 20A-20H. OSM induces Eda2r expression in muscle and the depletion of OSMR protects from muscle wasting. FIG. 20A, Mouse primary myotubes were treated with recombinant TNFα, IL-6, LIF, and OSM (250 ng/mL each). mRNA levels were determined by RT-qPCR (n=3). FIGS. 20B-20C, Mouse primary myotubes were treated with recombinant OSM and EDA-A2 (250 ng/mL each) for 48 hours. Myotubes, which were also treated with the GFP adenovirus, were visualized under the fluorescence microscope. The scale bar is 50 μm (FIG. 20B). Average myotube diameter was measured (n=3) (FIG. 20C). FIG. 20D, Mouse primary myotubes were treated with recombinant OSM (250 ng/mL for 48 hr) and EDA-A2 (100 ng/mL for 24 hr.). Changes in gene expression were determined by RT-qPCR (n=3). FIGS. 20E-20H, Mice were inoculated with LLC cells and sacrificed after 16 days (WT-LLC n=5, other groups n=6 mice). Carcass weight without the tumor mass (FIG. 20E) and tumor weight (FIG. 20F) were measured. Collected adipose tissues were weighed (FIG. 20G). mRNA levels of quadriceps muscle were tested by RT-qPCR (WT-LLC n=5, other groups n=6 mice) (FIG. 20H). The values are mean±SEM. Each dot represents a biological replicate. The data represent two (FIGS. 20A-20D) or three (FIGS. 20E-20H) independent experiments. Statistical analysis was conducted by using one-way ANOVA with Tukey's multiple-comparison test (FIG. 20A, and FIGS. 20C-20D) and two-way ANOVA with Tukey's multiple-comparison test (FIG. 20H).

FIGS. 21A-21D. NIK inhibition blocks EDA-A2 and TWEAK-induced muscle atrophy. Mouse primary myotubes were treated with SMI1 (5 μM), EDA-A2 (250 ng/mL) and TWEAK (250 ng/mL) for 48 hr. Protein expression was studied using western blotting (FIG. 21A and FIG. 21B). Myotubes were immunostained for myosin heavy chain protein. Nuclei were visualized using DAPI. Scale bar is 50 μm (FIG. 21C). Myotube dimeter was measured using Image J (FIG. 21D). Statistics by one-way ANOVA. *p<0.05, **p<0.01, ***p<0.001.

### DETAILED DESCRIPTION OF THE EMBODIMENTS

In the present invention, EDA2R-NIK and OSM-OSMR pathways are identified as drivers of muscle atrophy. The elements of these pathways, including EDA-A2, EDA2R, NIK, OSM, and OSMR, are expressed as follows.

Binding of EDA-A2 to its receptor EDA2R triggers a series of events, which consequently lead to the accumulation of NIK protein in the cell. NIK phosphorylates and activates IKKα, which in turn phosphorylates transcription factor p100-NFκB2. Phosphorylation of the p100 protein stimulates its processing into the active p52 form. p52-NFκB2 heterodimerizes with transcription factor Relb and this heterodimer enters the nucleus to regulate gene expression. In muscles cells, EDA2R/NIK signaling induces the transcription of atrophy genes, which are Atrogin1 and MuRF1, and non-canonical NFκB2 signaling elements, which are Nik, Nfkb2, and Relb. The depletion or inhibition of EDA-A2, EDA2R, and NIK prevents muscle atrophy in cultured myotubes or muscles of tumor-bearing mice.

Binding of OSM (Oncostatin M) to its receptor protein OSMR and co-receptor gp130 stimulates JAK1/2. Upon activation, these kinases autophosphorylate, and also phosphorylate the receptors, thereby causing STAT3 transcription factors to bind to the receptor. JAK1/2 subsequently phosphorylates and activates STAT3 proteins, which homodimerize and enter the nucleus to regulate gene expression. In muscle cells, OSM signaling leads to increased transcription of atrophy-related genes, including Atrogin1, Ampd3, Mt1/2, Eda2r, Sln, Cepbd, and Serpina3n. The depletion or inhibition of OSM and OSMR prevents muscle atrophy in cultured myotubes or muscles of tumor-bearing mice.

The present invention relates to the use of an EDA2R inhibitor or NIK inhibitor in the treatment of skeletal muscle atrophy. The present invention relates to the use of EDA2R inhibitor alone or NIK inhibitor alone or additionally in combination with the OSMR inhibitor in the treatment and/or prevention of skeletal muscle atrophy disease, in particular the treatment and/or prevention of skeletal muscle atrophy disease which is induced by tumor or linked to cancer cachexia.

The present invention also relates to the use of EDA2R inhibitor or NIK inhibitor as a drug in the treatment of skeletal muscle atrophy.

As used herein, the expression “pharmaceutically acceptable” refers to molecules or compounds which do not produce any adverse allergic effect or other undesirable reaction when administered to an animal or human.

As used herein, the term “excipient” refers to any pharmaceutically acceptable auxiliary substance, such as preservative, filler, lubricant, pH adjuster, disintegrant, surfactant, solvent, viscosity agent, emulsifier, and dispersant, which can be used in pharmaceutical formulation studies.

As used herein, the term “treatment” means the prevention or reduction or complete elimination of the occurrence or progression of the condition to which the term applies or one or more symptoms of the said condition. In the description of the invention, the term “preventive treatment” is included within the term treatment and used specifically to cover the prevention of the occurrence of one or more symptoms of the condition.

As used herein, the expression “therapeutically effective amount” refers to the dose of the drug which provides the specific pharmacological response for which the drug is administered to a patient in need of such treatment.

The present invention relates to a method of using EDA2R inhibitor or NIK inhibitor in the treatment of skeletal muscle atrophy in a subject. In the said method, a therapeutically effective amount of EDA2R inhibitor or NIK inhibitor is administered to treat skeletal muscle atrophy. A therapeutically effective amount of the inhibitor which inhibits the EDA2R-NIK signaling pathway is administered to the subject either alone or in combination with the inhibitor which inhibits the OSM-OSMR signaling pathway.

Embodiments of the method may include one or more of the following features. Here, the inhibitor can be a small molecule, an antibody, an antisense oligonucleotide, a small interfering RNA (siRNA), or a short hairpin RNA (shRNA). The said inhibitor may specifically and directly inhibit the EDA2R-NIK signaling pathway, or directly or indirectly inhibit the OSM-OSMR signaling pathway. The subject may suffer from a condition associated with muscle atrophy selected from a group such as cancer, kidney disease, heart failure, obstructive lung disease, diabetes, acquired immunodeficiency syndrome (AIDS) or from skeletal muscle atrophy associated with Cushing's syndrome, burn trauma, sepsis, aging-associated sarcopenia, Duchenne muscular dystrophy (DMD), Facioscapulohumeral muscular dystrophy (FSHD), Limb-girdle muscular dystrophy (LGMD), and amyotrophic lateral sclerosis (ALS).

The pharmaceutical formulations of the present invention can be prepared using standard techniques or production methods known in the state of the art.

The pharmaceutical formulation of the present invention is formulated to be used in the treatment of skeletal muscle atrophy. The present invention relates to a pharmaceutical formulation including a therapeutically effective amount of EDA2R inhibitor or NIK inhibitor as the active substance for use as a drug in the treatment of skeletal muscle atrophy. The said pharmaceutical formulation includes a therapeutically effective amount of the active substance and at least one pharmaceutically acceptable excipient.

In the first aspect, the use of an agent mentioned above generally is the use of an agent which inhibits the EDA2R-NIK signaling pathway or the OSM-OSMR signaling pathway in the production of a drug for preventing skeletal muscle atrophy.

The present invention also provides a method of treating a skeletal muscle atrophy patient, which includes administering a therapeutically effective amount of EDA2R inhibitor or NIK inhibitor to the patient.

The EDA2R inhibitor or NIK inhibitor which is the active substance according to the invention, can be administered by any route via known administration methods (oral, systemic, parenteral, nasal, enteral, etc.). The said inhibitor may be administered in combination with a second therapeutic agent, which may be a chemotherapeutic agent, a corticosteroid, an immunosuppressive agent, or an anti-inflammatory agent.

The quantities (therapeutically effective amounts to be received in one administration; dose amount) of the active substance of the present invention are adjusted so that they are effective in achieving the desired therapeutic response for a pharmaceutical form specific to the method of administration. Accordingly, dosages and excipients can be adjusted depending on the desired therapeutic effect, administration route, desired treatment duration, and other criteria.

The specific dose level for any patient (human or mouse) can be adjusted on a treatment-specific basis depending on several criteria, including body weight, general health, gender, diet, duration and route of administration, intestinal absorption and excretion characteristics, combination with other drugs, and severity.

In the preferred embodiment of the invention, the total daily dose of the said EDA2R inhibitor or NIK inhibitor for use in the treatment of skeletal muscle atrophy disease, which is administered in single dose or divided doses, may be in mg/kg body weight amounts at doses that can reach ng/mL levels in the blood daily.

In studies conducted within the scope of the present invention, it has been shown that EDA2R upregulation contributes to muscle loss during cancer cachexia. This study has demonstrated that noncanonical NFκB activation triggers muscle atrophy in which NIK kinase plays a central role. It has been shown that deletion of either EDA2R or NIK in mice was sufficient to provide resistance against tumor-induced muscle wasting. EDA2R expression is induced by inflammatory cytokines, and we identified OSM as an important regulator. In fact, the depletion of OSMR in muscle protected from the wasting of this tissue. Our results suggest that OSM/OSMR signaling acts in parallel to the EDA2R/NIK pathway and reinforces muscle atrophy and EDA2R upregulation.

There is an urgent need for an effective treatment against cachexia-associated muscle wasting. Our findings suggest that elements of EDA2R/NIK and OSM/OSMR pathways may serve as novel therapeutic targets. Pharmacologic inhibition of these pathways can prevent muscle loss. The blockade of EDA2R or OSMR and the inhibition of NIK kinase may be useful in reversing the muscle loss. Interestingly, the depletion of muscle OSMR was sufficient to both attenuate muscle loss and silence the EDA2R activation, making the OSM/OSMR pathway a potentially attractive therapeutic target.

Targeting of EDA2R-NIK and OSM-OSMR pathways will benefit patients who suffer from muscle loss due to cancer cachexia. However, this benefit is likely to have an impact on patients who suffer from muscle loss due to kidney disease, heart failure, obstructive lung disease, diabetes, acquired immunodeficiency syndrome (AIDS), Cushing's syndrome, burn trauma, sepsis, aging-associated sarcopenia, and muscular dystrophies, including Duchenne muscular dystrophy (DMD), Facioscapulohumeral (FSHD) and Limb-girdle (LGMD) muscular dystrophies, and neurodegenerative diseases, including, amyotrophic lateral sclerosis (ALS).

The following examples are to better illustrate the subject of the invention and the subject of the invention is not limited to these examples.

### EXAMPLES

One of our findings, which forms the basic foundations of the invention, demonstrate the upregulation of the Ectodysplasin A2 Receptor (EDA2R) in tumor-bearing mice and cachectic cancer patients by gene expression analysis in muscle tissues. Here, we show that activation of EDA2R signaling promotes skeletal muscle atrophy. Stimulation of primary myotubes with EDA-A2, which is the EDA2R ligand, triggered significant cellular atrophy by inducing the expression of the muscle atrophy-related genes, Atrogin1 and MuRF1. EDA-A2-induced myotube atrophy included activation of the noncanonical NFκB pathway and depended on NIK kinase activity. While overexpression of EDA-A2 promoted muscle wasting in mice, the deletion of EDA2R or muscle NIK protected tumor-bearing mice from the loss of muscle mass and function. Tumor-induced Oncostatin M (OSM) upregulated muscle EDA2R expression and muscle-specific Oncostatin M Receptor (OSMR) knockout mice were resistant to tumor-induced muscle loss. Our results show that EDA2R/NIK signaling mediates cancer-associated muscle atrophy in an OSM/OSMR-dependent manner. Thus, it has been demonstrated that therapeutic targeting of these pathways plays a role in the prevention of muscle loss.

### Example 1 EDA2R is Induced During Muscle Wasting

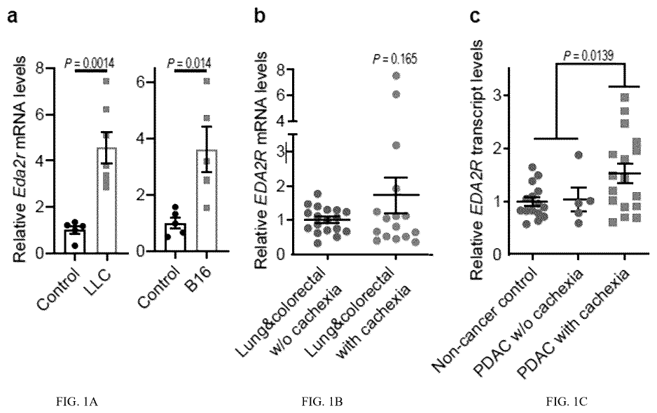

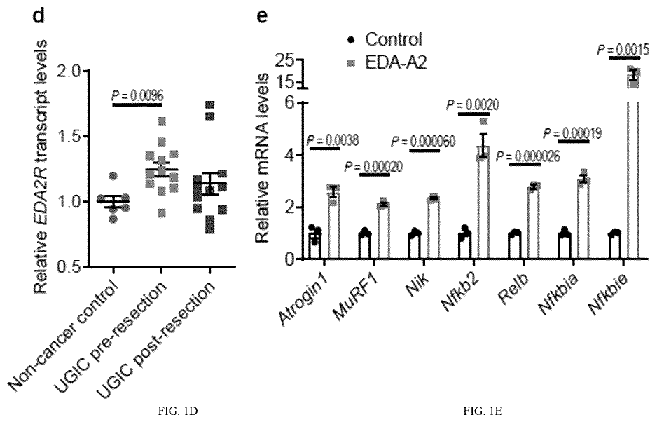

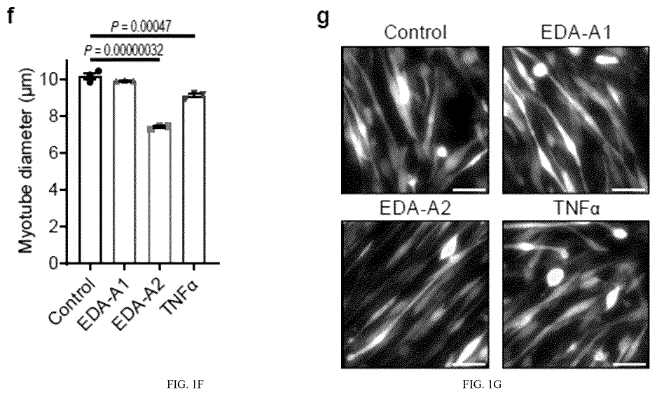

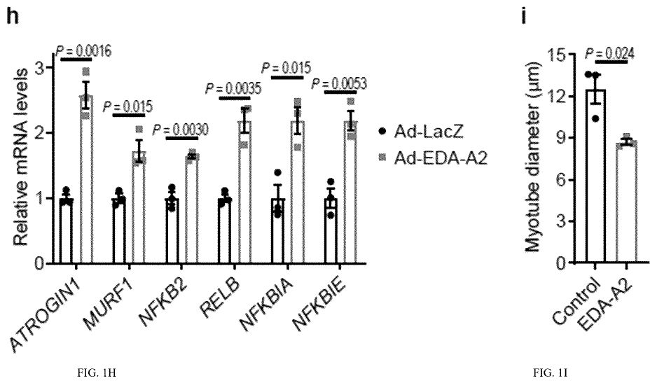

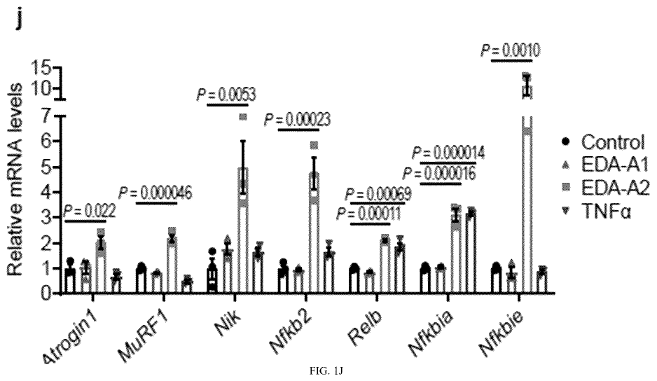

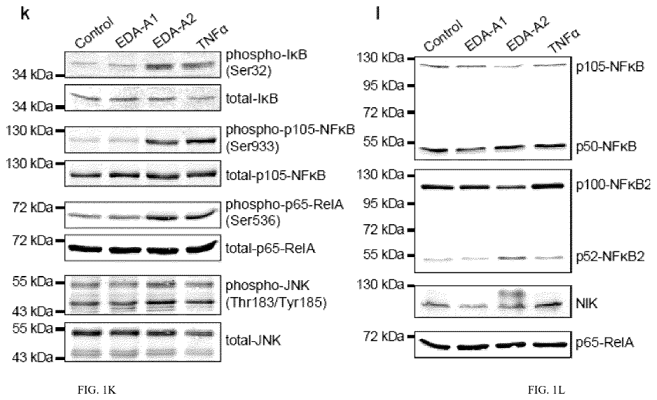

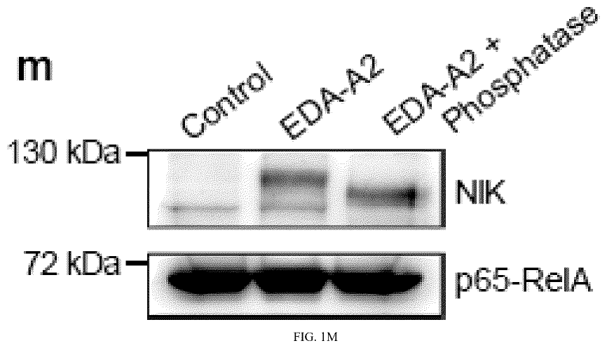

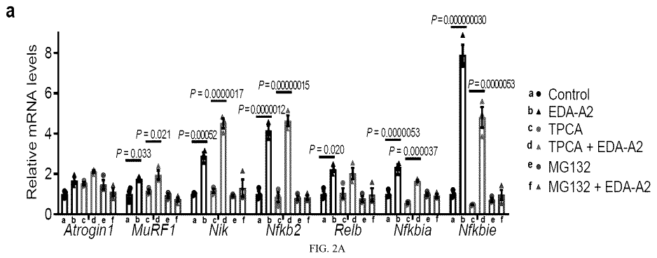

We investigated the skeletal muscle wasting, which is caused by cachexia-inducing Lewis Lung Carcinoma (LLC) and B16 melanoma tumors in the syngeneic C57BL/6 mice [9,10]. Our gene expression analysis demonstrated the significant upregulation of Eda2r mRNA in skeletal muscles of tumor-bearing mice (FIG. 1A). In fact, by comparing gene expression in different tissues of mice, we detected high levels of Eda-a1 and Eda-a2 mRNA in skeletal muscle (FIGS. 11A-11B). While Edar expression was at the highest level in the skin, mRNA levels of Eda2r were markedly enriched in skeletal muscle (FIGS. 11C-11D), suggesting a potential role for EDA2R signaling in muscle pathophysiology. By testing gene expression in muscle biopsies, we have detected high EDA2R mRNA in a subset of lung and colorectal cancer patients with cachexia (FIG. 1B). Furthermore, our analysis of gene expression datasets showed that EDA2R transcript levels were significantly increased in muscle biopsies of cachectic patients with pancreatic ductal adenocarcinoma (PDAC) compared to non-cachectic PDAC patients and non-cancer individuals (FIG. 1C). Similarly, muscle EDA2R levels were elevated in cachectic patients with upper gastrointestinal cancers (UGIC) compared to healthy controls. Upon the resection of tumors, there was a trend for reduced EDA2R expression in these patients (FIG. 1D and FIG. 12A). In addition, we also detected significantly high EDA2R transcript in muscle biopsies of Duchenne muscular dystrophy (DMD) and Facioscapulohumeral muscular dystrophy (FSHD) patients with reduced muscle mass and function (FIGS. 12B-12D). In light of these observations, we questioned whether the EDA-A2/EDA2R pathway plays a role in muscle loss.

### Example 2 Activation of EDA2R Promotes Muscle Atrophy

First, we examined the outcome of the activation of this pathway in muscle cells. For this purpose, we isolated primary myoblasts from C57BL/6 mice and differentiated them into fully mature myotube cells. Treatment of primary myotubes with recombinant EDA-A2 protein stimulated mRNA levels of muscle atrophy-related genes, Atrogin1 (Fbxo32) and MuRF1 (Trim63) (FIG. 1E). These genes encode E3 ubiquitin ligase enzymes, which are well-known inducers of muscle protein breakdown2. EDA-A2 treatment promoted cellular atrophy in primary myotubes, as evidenced by a reduction in the diameter of these cells (FIGS. 1F-1G). While primary myotubes did not respond to EDA-A1 treatment, a marginal effect on cellular atrophy was induced by TNFα (FIGS. 1F-1G). Overexpression of EDA-A2 and not EDA-A1 in primary myotubes promoted mRNA levels of Atrogin1 and MuRF1 and induced cellular atrophy (FIGS. 13A-13C). In addition, the overexpression of EDA-A2 or the administration of recombinant EDA-A2 also induced ATROGIN1 and MURF1 expression in human myotubes and led to a reduction in myotube diameter (FIGS. 1H-1I and FIGS. 13D-13F). We further documented the atrophic effects of EDA-A2 in mouse primary myotubes by measuring the protein levels of myosin heavy chain (MyHC). Immunofluorescently labeled MyHC signal was significantly reduced after EDA-A2 administration (FIGS. 13G-13H). Downregulation of MyHC was also detected by western blotting. In particular, proteasomal inhibition by MG132 reversed this effect, suggesting that EDA-A2 promoted MyHC loss by increasing proteasomal degradation (FIG. 13I).

### Example 3 NFκB Pathway at the Downstream of EDA2R

Previous studies have shown that NFκB signaling is activated by EDA-A2/EDA2R [11,12]. Therefore, we further tested the changes in mRNA and protein levels of NFκB factors and IκB inhibitors thereof. NFκB transcription factors are normally sequestered in the cytoplasm by inhibitory IκB proteins. The canonical NFκB signaling includes IKKβ-dependent phosphorylation and degradation of IκBs, while the noncanonical (alternative) NFκB activation depends on NIK, which promotes the processing of p100-NFκB2 into the active p52-NFκB2 form, resulting in nuclear translocation of the p52-NFκB2/RelB complex [13]. In particular, administration or overexpression of EDA-A2 increased mRNA levels of Nik (Map3k14), Nfkb2, Relb, Nfkbia, and Nfkbie in mouse primary myotubes (FIG. 1E and FIG. 13A and FIGS. 14A-14B). A similar effect was also detected in human myotubes (FIG. 1H and FIG. 13E). Compared to TNFα, EDA-A2 treatment in mouse primary myotubes produced a more significant effect on the expression of the atrophy genes and the NFκB signaling elements, while EDA-A1 failed to stimulate these changes (FIG. 1J). Our results show that EDA-A2 induces the atrophy of myotubes and the transcription of noncanonical NFκB signaling components with high levels of IκBs.

Next, we examined the activation of the canonical NFκB pathway by determining the phosphorylation of IκB, p105-NFκB, and p65-RelA. Treatment of mouse primary myotubes with either EDA-A2 or TNFα acutely induced the phosphorylation of these proteins (FIG. 1K). We also detected an EDA-A2-induced increase in the phosphorylation of JNK, which was previously included in EDA2R signaling [11,14] (FIG. 1K). After 24 hours of treatment, no stable change was detected in the processing of p105-NFκB into p50-NFκB (FIG. 1L). Interestingly, prolonged treatment of primary myotubes with recombinant EDA-A2 promoted alternative activation of NFκB signaling, as evidenced by increased processing of p100-NFκB2 into p52-NFκB2, which is a process driven by NIK13. In fact, EDA-A2 treatment elevated the protein levels of NIK and particularly caused an electrophoretic mobility shift of this protein (FIGS. 1L-1M). This shift is partly due to the phosphorylation of NIK, since it can be partially suppressed by alkaline phosphatase treatment (FIG. 1M). Other types of post-translational modifications also likely contribute to this behavior. Wild-type mouse NIK protein, which is overexpressed in primary myotubes, also exhibited mobility shift, unlike kinase-dead and autophosphorylation-deficient NIK mutants (FIG. 14C). In particular, EDA-A2 treatment increased protein levels of the mutants without causing a shift. Therefore, EDA-A2-induced mobility shift likely requires intact NIK kinase activity and depends on the autophosphorylation of the protein (FIG. 14C). This shift was not detected for human NIK protein, which is overexpressed in mouse primary myotubes (FIG. 14D). In addition, overexpression of EDA-A2 in primary myotubes also activated the alternative NFκB signaling through NIK accumulation (FIG. 14E). Our results show that EDA-A2 is a potent inducer of atrophy in myotubes and leads to transient and stable activation of the canonical and noncanonical NFκB pathways, respectively.

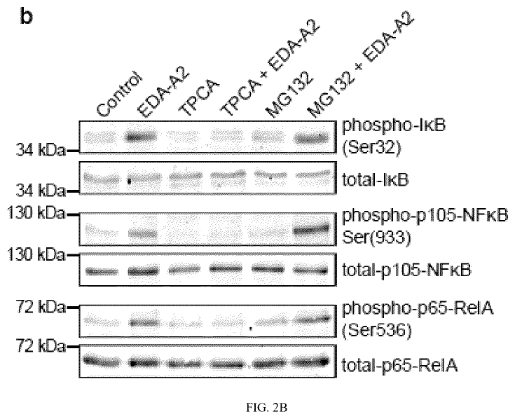

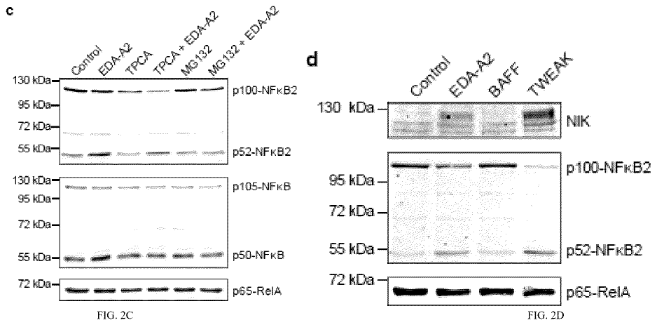

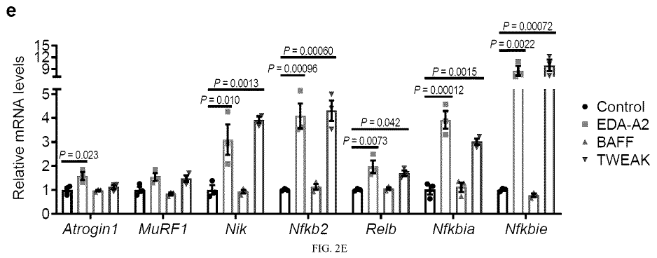

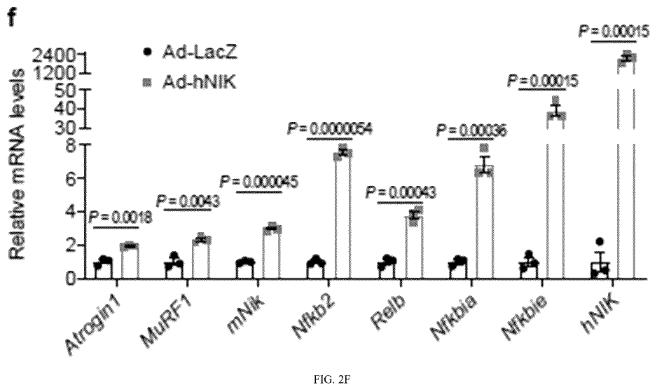

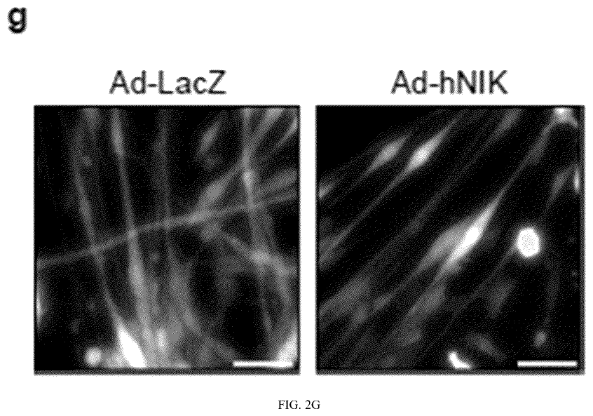

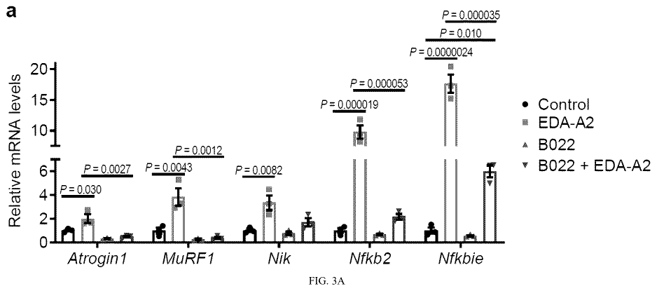

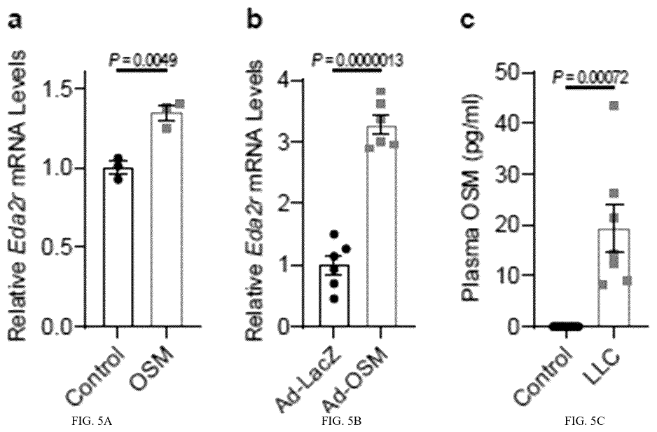

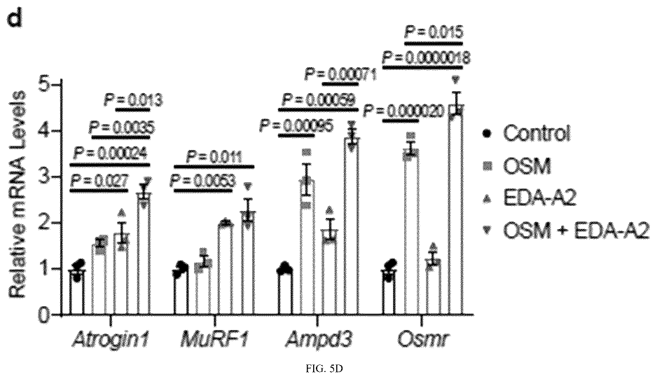

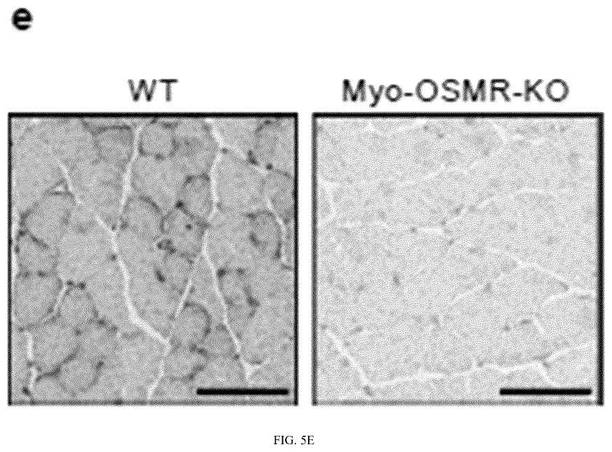

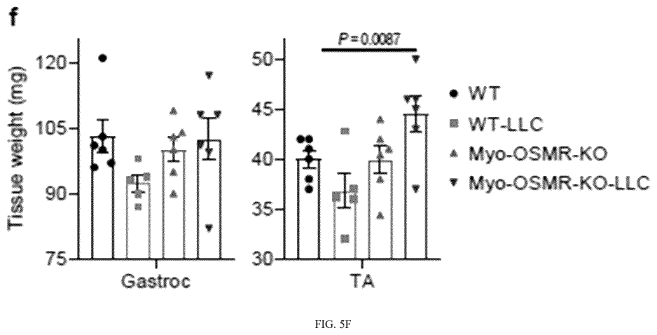

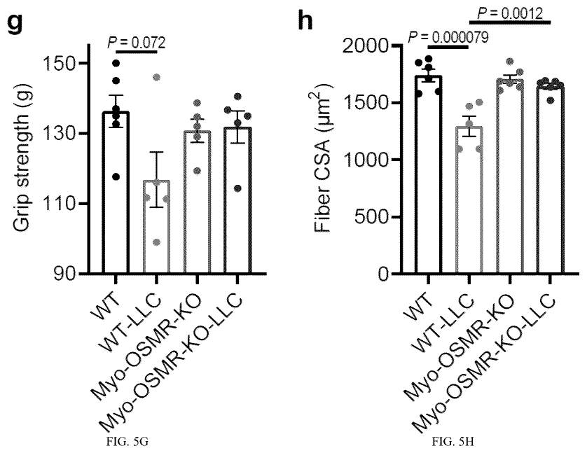

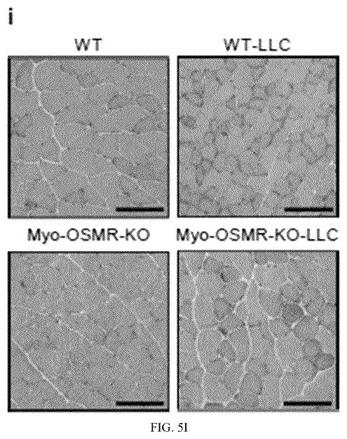

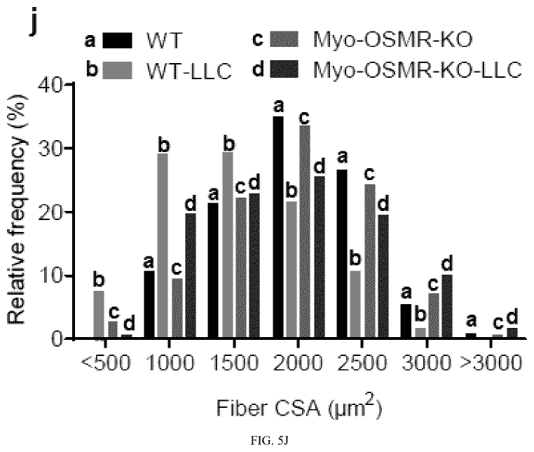

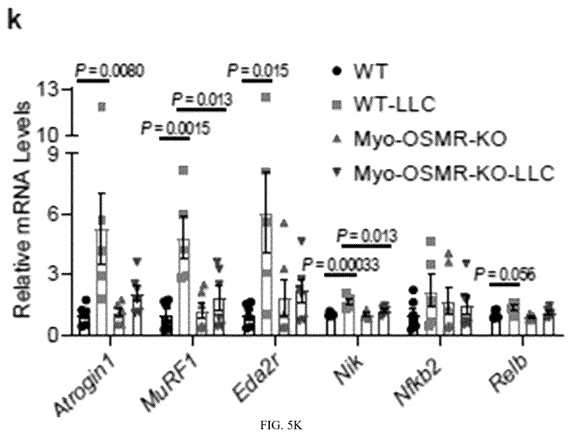

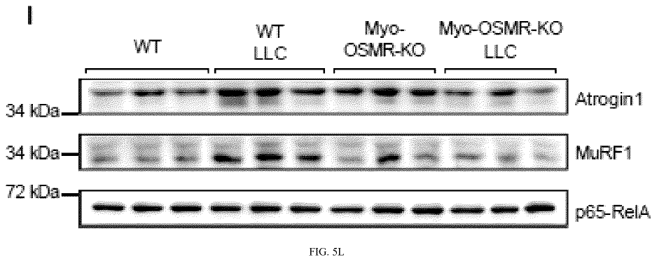

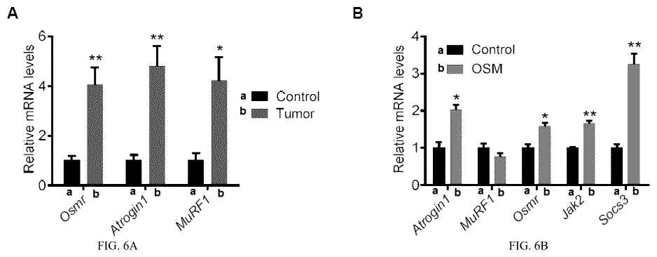

In order to distinguish the relative contribution of NFκB pathways to EDA-A2-induced atrophy, we treated primary myotubes with the selective IKKβ inhibitor TPCA-1 and the proteasome inhibitor MG132. We found that EDA-A2-induced phosphorylation of IκB, p105-NFκB, and p65-RelA was blocked by TPCA-1 treatment. However, inhibition of the canonical NFκB pathway did not suppress the effects of EDA-A2 on gene expression (FIGS. 1L-1M and FIGS. 2A-2B). We tested additional IκB phosphorylation inhibitors such as BAY 11-7082 and BOT-64, which also failed to block EDA-A2-induced transcriptional changes (FIG. 15A). IκB phosphorylation inhibitors also did not change p100-NFκB2 processing driven by EDA-A2 (FIG. 2C and FIG. 5B). On the other hand, proteasomal inhibition by MG132 was able to prevent p100-NFκB2 processing and the transcriptional effects elicited by EDA-A2 treatment in primary myotubes (FIG. 2A and FIG. 2C), consistent with an implication of the alternative NFκB activation at the downstream of EDA-A2 signaling.

Since EDA-A2 induced mRNA and protein levels of NIK, we compared it with cytokines which are known to activate NIK kinase, such as BAFF and TWEAK [15]. While BAFF failed to trigger an effect in primary myotubes, EDA-A2 and TWEAK acted similarly as both factors stimulated the mRNA expression of the target genes, the accumulation of NIK protein, and the processing of p100-NFκB2 (FIGS. 2D-2E). In fact, TWEAK has previously been associated with muscle atrophy [16,17]. Our findings suggest that both factors may utilize a similar downstream signaling mechanism in myotubes.

### Example 4 Activation of NIK Triggers Muscle Atrophy

If noncanonical NFκB signaling plays a role in muscle atrophy, then activation of this pathway alone should stimulate this process. For this purpose, we transduced primary myotubes with an adenovirus expressing NIK. The overexpression of NIK promoted the processing of NFκB2 and the expression of Atrogin1, MuRF1, and other EDA-A2 targets (FIG. 16A and FIG. 2F). In fact, overexpression of NIK was sufficient to induce cellular atrophy in primary myotubes (FIG. 2G and FIG. 16B). Similar effects on gene expression and cellular atrophy were also observed in human myotubes (FIGS. 16C-16E). We overexpressed mutant mouse NIK isoforms in mouse primary myotubes. While the kinase-dead mutant did not elicit an effect, the autophosphorylation-deficient mutant triggered partial responses in the NFκB2 processing and target gene expression (FIGS. 16F-16G).

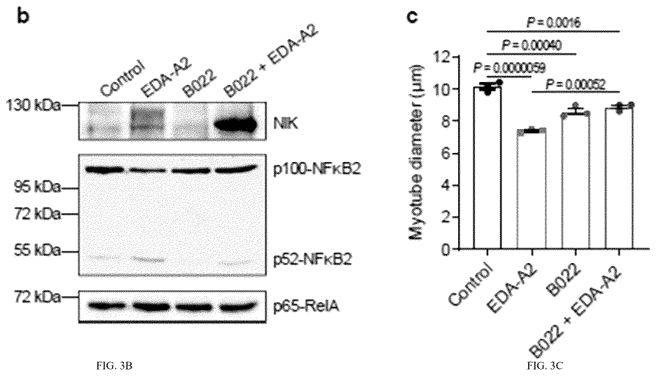

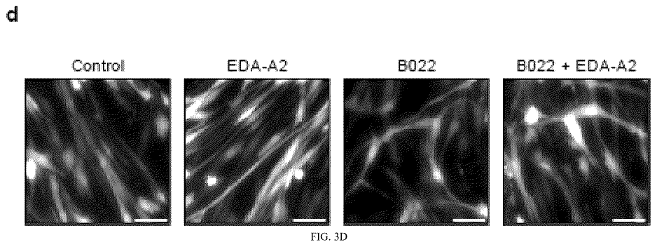

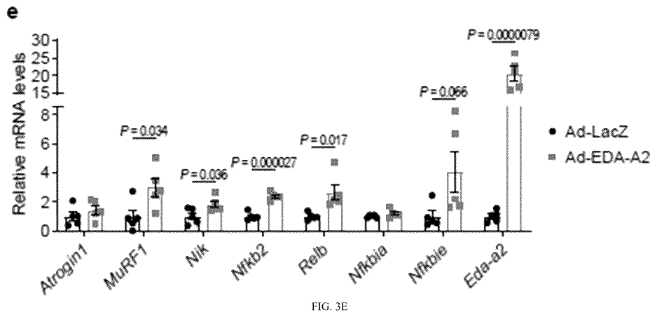

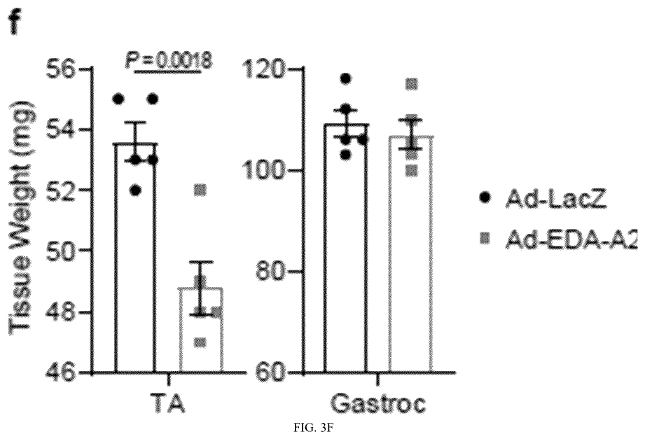

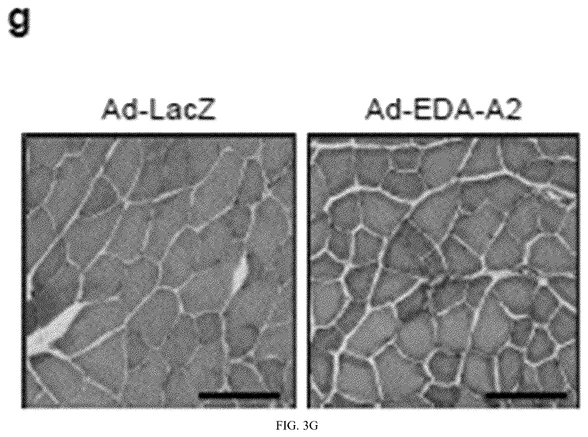

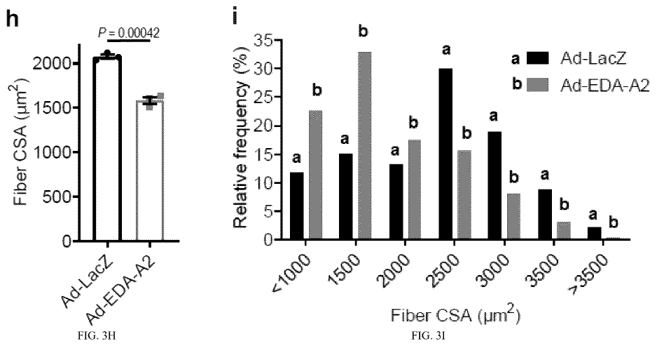

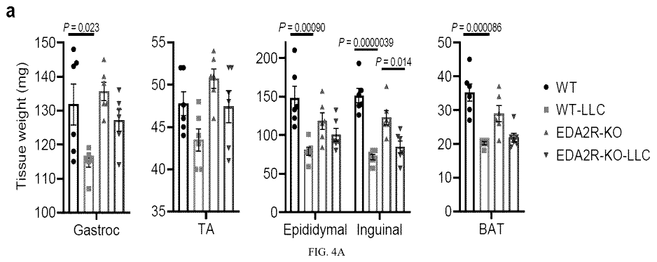

Then, we reviewed the necessity of the alternative NFκB activation for EDA-A2-induced myotube atrophy. Treatment of primary myotubes with B022, which is a specific NIK kinase inhibitor [18,19], blocked NIK-induced NFκB2 processing and gene expression in a dose-dependent manner (FIGS. 17A-17B). Combined treatment with B022 and recombinant EDA-A2 also inhibited the expression of Atrogin1, MuRF1, and other EDA-A2 target genes and blunted the NFκB2 processing in primary myotubes (FIGS. 3A-3B). In fact, the B022 treatment also blocked the EDA-A2-induced mobility shift of NIK protein, while the original NIK signal was greatly increased, possibly due to a negative feedback loop broken by the inhibition. A similar effect on NIK protein levels was also observed when EDA-A2-overexpressing primary myotubes were treated with B022 (FIG. 17C). In addition, overexpression of a dominant-negative NIK form which suppressed the NIK-induced processing of NFκB2 also interfered with the upregulation of atrophy genes by EDA-A2 (FIGS. 17D-17F). Upon NIK kinase inhibition, EDA-A2 failed to stimulate atrophy in primary myotubes, indicating that NIK signaling plays an important role in EDA-A2-induced atrophy (FIGS. 3C-3D).

### Example 5 Overexpression of EDA-A2 Leads to Muscle Loss

The potent effects of EDA-A2 on primary myotubes encouraged us to study its induction in muscle tissue. Previously, transgenic mice overexpressing EDA-A2 in skeletal muscle were generated. These mice exhibited profound muscle degeneration, which was prevented by the deletion of EDA2R8. Here, we acutely overexpressed EDA-A2 by adenoviral delivery in the tibialis anterior (TA) muscle of mice. Within 7 days, the expression of EDA-A2 target genes, including MuRF1, Nik, Nfkb2, and Relb, were induced in TA muscles (FIG. 3E). In parallel, the weight of TA muscles transduced with EDA-A2 adenovirus was significantly reduced, while the weight of untreated gastrocnemius muscles remained similar (FIG. 3F). Hematoxylin and eosin (H&E) staining of TA muscles showed that muscle fiber cross-sectional area reduces and the frequency of fibers with small cross-sectional area increases in response to the overexpression of EDA-A2 (FIGS. 3G-3I). These results suggest that EDA-A2 induction can promote muscle atrophy in vivo.

### Example 5 Tumor-Induced Muscle Loss Requires EDA2R

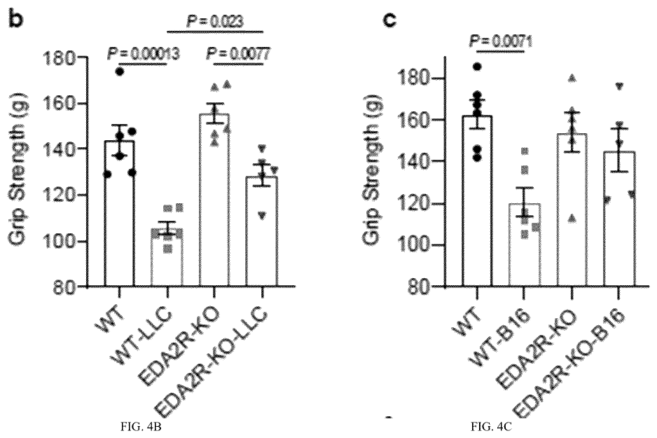

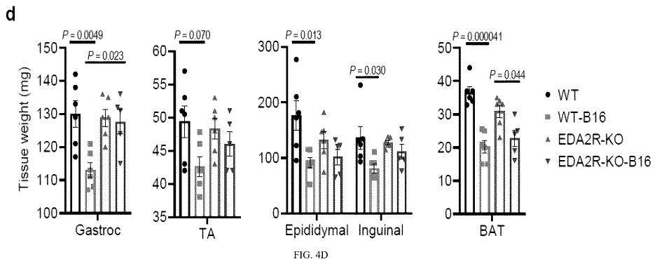

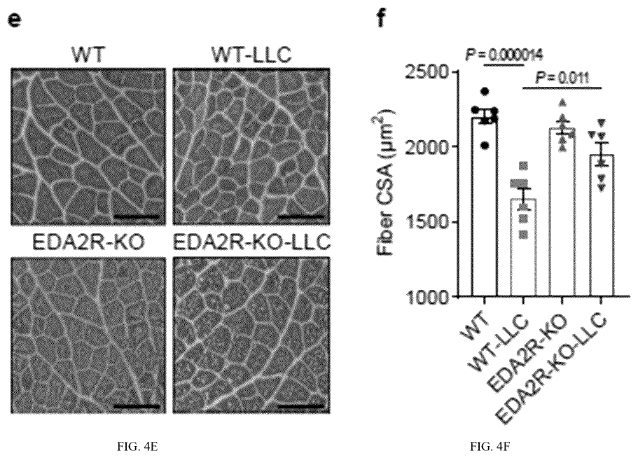

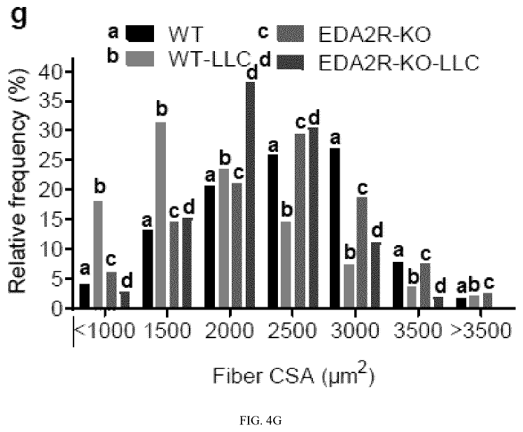

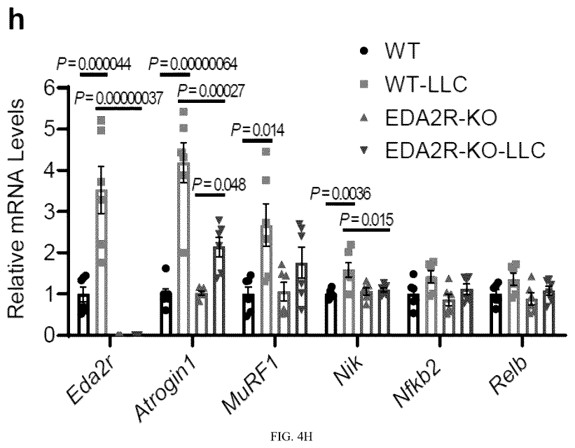

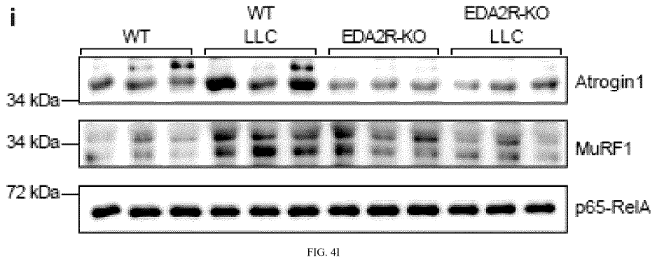

Next, we reviewed the role of EDA2R/NIK signaling in tumor-induced muscle wasting. We utilized EDA2R-null (EDA2R-KO) mice with normal body weight and without any obvious phenotypic characteristics8. We inoculated littermate wild-type and knockout mice with LLC tumors. Muscle wasting was remarkably reduced in EDA2R-KO mice, as evidenced by the preservation of gastrocnemius and TA muscles (FIG. 4A). Accordingly, these mice had higher tumor-free body weight compared to tumor-bearing wild-type mice (FIGS. 18A-18B) and also exhibited improved muscle performance, as measured by forelimb grip strength (FIG. 4B). Tumor weight, the expression of immune response-related genes in tumors, and plasma C-Reactive Protein (CRP) levels as an indicator of systemic inflammation were comparable between the wild-type and knockout groups (FIGS. 18B-18D). We also dissected and weighed adipose tissue depots, such as epididymal and inguinal white adipose tissue and interscapular brown adipose tissue (BAT). However, no significant effect was detected on adipose tissue loss (FIG. 4A). Similar results were obtained on tumor-free body weight, muscle mass, and physical strength when EDA2R-KO mice were inoculated with B16 tumors (FIGS. 4C-4D and FIGS. 18E-18F).

The improvements in muscle mass and function had also an impact on muscle histology. H&E staining of muscle tissue showed that there was an increase in muscle fiber cross-sectional area in tumor-bearing knockout mice compared to wild-type counterparts (FIGS. 4E-4F, FIGS. 18G-18H). Tumor-induced enrichment of muscle fibers with a small cross-sectional area was suppressed in EDA2R-deficient mice (FIG. 4G and FIG. 18I). We also examined changes in the expression of EDA-A2 target genes in these samples. In the absence of EDA2R, tumor-induced mRNA expression of Atrogin1, MuRF1, and Nik was suppressed, while a limited induction in mRNA levels of Nfkb2 and Relb was detected (FIG. 4H and FIGS. 18J-18L). In addition, protein levels of Atrogin1 and MuRF1 were also reduced in the muscles of tumor-bearing EDA2R-KO mice (FIG. 4I). These findings show that EDA2R function is required for tumor-induced muscle loss.

### Example 6 Nik Plays a Role in Muscle Wasting

In order to identify this pathway in more detail, we generated skeletal muscle-specific NIK knockout mice (Myo-NIK-KO). We confirmed that the deletion was restricted to skeletal muscle by comparing mRNA levels of Nik in various tissues (FIG. 19A). Then, we inoculated these mice with LLC tumors and examined their cachexia phenotype. Similar to EDA2R-KO mice, Myo-NIK-KO mice were also resistant to tumor-induced weight loss (FIGS. 19B-19C). The lack of NIK in muscles of tumor-bearing mice prevented muscle loss and also preserved muscle function, as determined by the measurements of forelimb grip strength (FIGS. 9D-9E). No significant effect was observed on adipose tissue loss (FIG. 9D). The examination of muscle histology of tumor-bearing mice also showed that there was an increase in muscle fiber cross-sectional area and a decrease in the frequency of fibers with small cross-sectional area in Myo-NIK-KO mice compared to wild-type counterparts (FIGS. 19F-19H). Analysis of gene expression in muscle tissues also showed that there was a reduction in tumor-induced mRNA and protein levels of Atrogin1 and MuRF1 in the knockout mice (FIGS. 19I-19K). The similarities between Myo-NIK-KO and EDA2R-KO mice in terms of the tumor-induced responses show that a common pathway including EDA2R/NIK acts to promote muscle atrophy.

### Example 7 EDA2R is Upregulated by Oncostatin M

We also investigated how tumors upregulate Eda2r expression in muscle tissue. By testing various tumor-induced cytokines on primary myotubes, we observed the upregulation of Eda2r by Oncostatin M (OSM), which is an IL-6 family cytokine involved in a variety of biological processes, including muscle atrophy (FIG. 5A and FIG. 20A) [20,21]. OSM significantly increased Eda2r mRNA when overexpressed in TA muscles of mice (FIG. 5B). In the analysis of blood plasma from mice bearing LLC tumors, high levels of OSM were detected (FIG. 5C), indicating that the tumor-induced OSM may activate EDA2R signaling in muscle tissue. In fact, treatment of mouse primary myotubes with recombinant OSM protein also stimulated the expression of Atrogin1 and the resultant atrophy, indicating that OSM itself is an atrophy-inducing factor (FIG. 5D and FIGS. 20B-20C). We detected that combined treatment of OSM and EDA-A2 led to additive atrophy-related gene expression in primary myotubes. After testing changes in mRNA levels of OSM target genes, Osmr and Socs3; EDA-A2-specific targets, MuRF1, Nik, Nfkb2, and Relb; and OSM/EDA-A2 common gene targets, Atrogin1 and Ampd3, we detected additional effects on the expression of Atrogin1, Ampd3, and Osmr (FIG. 5D and FIG. 20D). The combination of OSM and EDA-A2 also caused a greater reduction in myotube diameter (FIGS. 20B-20C). These secreted factors can operate alone or together to induce the atrophy of cultured myotubes.

### Example 8 Tumor-Induced Muscle Loss Requires OSMR

In order to determine the role of OSM in EDA2R regulation and muscle wasting, we generated skeletal muscle-specific OSM receptor knockout (Myo-OSMR-KO) mice. We confirmed that OSMR was depleted in muscle fibers by using immunohistochemistry (FIG. 5E). Upon LLC tumor inoculation, Myo-OSMR-KO mice were protected from weight loss and muscle wasting (FIG. 5F and FIGS. 20E-20F). However, tumor-bearing knockout mice still lost adipose tissue mass (FIG. 20G). Muscle strength, which was assessed by the measurements of forelimb grip, reflected the preservation of muscle mass in the knockout mice, while the wild-type mice exhibited significant reduced performance (FIG. 5G). H&E staining of gastrocnemius tissue sections showed that tumor-bearing Myo-OSMR-KO mice had significantly wider muscle fibers than tumor-bearing controls (FIGS. 5H-5I). Fibers with small cross-sectional area were enriched in the latter group (FIG. 5J). Gene expression analysis showed that tumor-induced mRNA and protein levels of Atrogin1 and MuRF1 were reduced in the muscles of Myo-OSMR-KO mice compared to their wild-type counterparts (FIGS. 5K-5L and FIG. 20H). More importantly, depletion of OSMR also suppressed the upregulation of Eda2r and its downstream targets in these samples (FIG. 5K and FIG. 20H). These findings suggest that the OSM/OSMR pathway plays a major role in tumor-induced muscle wasting, including the activation of EDA2R/NIK signaling.

### Example 9 OSM Promotes Cellular Atrophy in Cultured Primary Myotubes

By utilizing the murine Lewis Lung Carcinoma (LLC) model of cancer cachexia, we detected that mRNA levels of the Osmr gene were highly elevated in atrophying muscles (FIG. 6A). Similarly, the expression of E3 ubiquitin ligase genes, Atrogin1 and MuRF1, whose protein products are well known to activate protein breakdown associated with muscle atrophy, was also elevated. In order to test whether increased OSMR activity contributes to the muscle atrophy process, we isolated mouse primary myoblast cells and differentiated them into myotubes. Treatment of the primary myotubes with a recombinant OSM protein induced expression of Atrogin1 without altering MuRF1 levels (FIG. 6B). OSM treatment also increased mRNA levels of its receptor Osmr and mRNA levels of the downstream components of cytokine signaling, which are Janus Kinase 2 (Jak2) and Suppressor of Cytokine Signaling 3 (Socs3) (FIG. 6B). In addition, OSM-treated myotubes exhibited reduced diameter, which is an indicator of cellular atrophy (FIG. 6C). Since myotube diameters were more significantly reduced compared to IL-6 and LIF, other IL-6 family cytokines involved in muscle atrophy, OSM promoted a stronger atrophy-inducing effect (FIGS. 6C-6D).

We investigated the effect of these cytokines on the global gene expression profiles of mouse primary myotubes by using RNA sequencing. It was observed that OSM had a larger footprint on the transcriptome of myotubes, as visualized in the heatmap of the differentially expressed genes (FIG. 6E). The datasets identifying these gene sets can be found in Gene Expression Omnibus (GEO) database with accession number GSE222208. From the analysis of RNA sequencing data, it was found that several muscle atrophy-related genes, including Atrogin1 (Fbxo32), Ampd3, Mt1, Mt2, Sln, Cebpd, Igfbp3, and Serpina3n, were upregulated (FIG. 6E). We tested mRNA levels of these genes in mouse primary myotubes which were treated with OSM, IL-6, or LIF. In parallel with the effect on the myotube appearance, OSM treatment promoted a significantly larger increase in Atrogin1 levels (FIG. 6F). Similarly, more significant changes in the expression levels of Ampd3, Mt1, Mt2, Igfbp3, and Serpina3n were detected in response to OSM treatment (FIG. 6F). Our findings suggest that OSM is a potent inducer of cellular atrophy in primary myotubes and significantly alters myotube gene expression.

### Example 10 OSM Utilizes JAK/STAT3 Signaling to Elicit its Effects in Myotubes

It is known that OSM/OSMR signaling activates STAT transcription factors and it has been previously reported that the JAK/STAT pathway is involved in atrophying muscle tissue [22, 23]. Therefore, we investigated the effect of OSM administration on the phosphorylation and activation of the JAK/STAT signaling components. Treatment of mouse primary myotubes with recombinant OSM induced the phosphorylation of JAK2, STAT1, STAT3, and STAT5 (FIG. 7A). Similar responses were obtained when myotubes were treated with recombinant LIF, while IL6 treatment elicited milder effects. OSM-induced phosphorylation events were completely blocked when myotubes were also treated with JAK1/2 kinase inhibitor Ruxolitinib (FIG. 7B). Accordingly, OSM-induced changes in mRNA levels of atrophy-related genes were also reversed by Ruxolitinib treatment, suggesting an indispensable role for JAK kinases at downstream of the OSM signaling (FIG. 7C). Activation of the NFκB signaling was also previously reported in the atrophying muscles [24].

The depletion of muscle-specific STAT3 was previously shown to attenuate tumor-driven muscle loss and STAT3 involvement in Atrogin1 transcription was reported [23, 25]. Therefore, we tested the role of STAT3 in OSM-induced atrophy by using a dominant-negative STAT3 isoform. STAT3-Y705F mutant was overexpressed in mouse primary myotubes by adenoviral delivery. The dominant-negative form blocked OSM-induced phosphorylation and activation of endogenous STAT3 protein (FIG. 7D). More importantly, OSM-induced changes in the expression of atrophy-related genes were suppressed by the overexpression of dominant-negative STAT3 (FIG. 7E). These myotubes were also resistant to OSM-induced cellular atrophy since the myotube diameter did not change (FIGS. 7F-7G). These findings indicate that OSM utilizes the JAK/STAT3 signaling to promote the expression of atrophy genes and the resultant cellular atrophy in primary myotubes.

### Example 11 Overexpression of OSM Causes Muscle Atrophy in Mice

We investigated the potential of OSM to promote muscle atrophy in vivo by overexpressing it in the tibialis anterior (TA) muscles of mice. For this purpose, we generated an adenoviral vector which expresses mouse OSM. OSM adenovirus was unilaterally administered to the TA muscle, while the contralateral TA muscle was transduced with a control LacZ adenovirus. The weight of TA muscles was significantly reduced 7 days after the transduction with Adeno-OSM, while gastrocnemius muscles from the same animals were not affected (FIG. 8A). Hematoxylin and eosin (H&E) staining of TA tissue sections showed that there was a significant decrease in muscle fiber cross-sectional area in response to Adeno-OSM (FIGS. 8B-8C). An increase in the frequency of fibers with a smaller cross-sectional area was detected (FIG. 8D). The overexpression of OSM in muscle tissue induced mRNA levels of Osmr, and the atrophy-related genes, which are Atrogin1, Ampd3, Cebpd, Igfbp3, Sln, Mt1, Mt2, and Serpina3n (FIG. 8E).

The overexpression of OSM also increased total protein levels of Atrogin1, MURF1, STAT1, STAT3, and STAT5 in muscle tissue (FIGS. 8F-8G). However, phosphorylation of only STAT3 was stimulated by OSM, based on the total protein levels (FIGS. 8F-8G). These results suggest that activation of the OSM/OSMR signaling can promote atrophy in muscle tissue in vivo.

### Example 12 Neutralization of OSM Ameliorates Tumor-Induced Muscle Wasting

In order to investigate the therapeutic potential of targeting OSM signaling for preventing cachexia-linked muscle loss, we utilized a neutralizing anti-OSM antibody. When administered to primary myotubes together with the recombinant OSM protein, the anti-OSM antibody prevented the upregulation of OSM target genes (FIG. 9A). After documenting its neutralizing effect, we administered the anti-OSM antibody to LLC-tumor bearing mice. A non-tumor-bearing group and a control tumor-bearing group received an isotype control IgG antibody. The antibody treatment was performed 10, 12, 14 and 15 days after tumor inoculation and mice were sacrificed 1 day after the last injection (FIG. 9B). Anti-OSM antibody did not affect the size of LLC tumors (FIG. 9C). Remarkably, the muscle mass of the tumor-bearing mice was preserved after anti-OSM administration (FIG. 9D), and these mice exhibited improved forelimb grip strength (FIG. 9E). H&E staining of gastrocnemius muscle sections also showed that muscle fiber cross-sectional area in the anti-OSM group was increased compared to the IgG group of the tumor-bearing mice (FIGS. 9F-9G). Tumor-induced increase in the frequency of muscle fibers with a small cross-sectional area was suppressed by anti-OSM administration (FIG. 9H). The neutralization of OSM also reduced the phosphorylation of STAT3 and the accumulation of Atrogin1 and MURF1 proteins in muscle tissue (FIGS. 9I-9J). These findings indicate that OSM plays a direct role in tumor-induced muscle loss and that the blockade of OSM activity can be used to ameliorate cachexia-associated muscle loss.

### Example 13 OSM Target Genes are Upregulated in Muscles of Cancer and Muscular Dystrophy Patients

We examined whether the activation of OSM/OSMR signaling is linked to muscle loss in humans. We analyzed publicly available human gene expression datasets and tested transcript levels of OSM target genes. Our analysis demonstrated that OSMR expression increased in muscle biopsies of pancreatic ductal adenocarcinoma (PDAC) patients who lost weight. Judge et al. compared gene expression profiles of rectus abdominis muscle biopsies from 17 cachectic PDAC patients, 5 non-cachectic PDAC patients, and 16 non-cancer controls by using microarrays [26]. Analysis of this dataset demonstrated that OSMR expression increased in cachectic patients compared to non-cachectic and non-cancer controls (FIG. 10A). We further analyzed this dataset in order to determine whether the expression of the gene targets of OSM/OSMR signaling correlates with OSMR transcript levels. We compiled the top 200 genes, which were significantly upregulated by OSM in primary myotubes, and performed gene set enrichment analysis (GSEA) by using this gene list. Annotated genes with known human orthologues were chosen. GSEA analysis of the datasets demonstrated that OSM target genes were significantly over-represented in muscle biopsies of cachectic PDAC patients (GSE130563; normalized enrichment score (NES)=2.17, P-value<0.001) (FIG. 10B).

Our gene expression analysis also demonstrated that OSMR transcript levels significantly increased in muscular dystrophy diseases, including Duchenne muscular dystrophy (DMD) and Facioscapulohumeral muscular dystrophy (FSHD). In a study by Dadgar et al., 49 muscle biopsies from patients with DMD, Becker muscular dystrophy (BMD) and limb girdle muscular dystrophy (LGMD) were investigated [27]. Analysis of this dataset (GSE109178) showed that OSMR transcript was upregulated in patients with DMD (n=17, fold change=4.0, Padj<0.0000009), BMD (n=11, fold change=3.1, Padj<0.0022), and dysferlin-deficient LGMD2B (n=8, fold change=4.5, Padj<0.009) compared to normal subjects (n=6) (FIG. 10C). The same research group also reported comparative profiling of 117 muscle biopsies from 13 different muscle disease groups. Analysis of the dataset (GSE3307) showed that OSMR was upregulated in patients with DMD (n=10, fold change=2.3, Padj<0.000014), BMD (n=5, fold change=2.2, Padj<0.018), acute quadriplegic myopathy (AQM) (n=5, fold change=2.4, Padj<0.0019), FSHD (n=14, fold change=1.6, Padj<0.008), juvenile dermatomyositis (JDM) (n=21, fold change=2.2, Padj<0.0007), calpain3-deficient LGMD2A (n=10, fold change=2.0, Padj<0.0007), dysferlin-deficient LGMD2B (n=11, fold change=2.7, Padj<0.0007), and fukutin-related protein (FKRP)-deficient LGMD2I (n=7, fold change=1.7, Padj<0.05) compared to normal subjects (n=18) (FIG. 10D). In addition, analysis of gene expression profiles of muscle biopsies from 10 DMD patients and 10 unaffected subjects studied by Haslett et al. (GSE1007) [28] demonstrated a 2.4-fold increase in OSMR levels in DMD patients (Padj<0.0002) (FIG. 10E). Lastly, we analyzed the dataset created by Wang et al., who collected MRI-informed muscle biopsies from FSHD patients and investigated the expression of Double homeobox 4 (DUX4) target genes whose aberrant expression in muscle is linked to this disease [29]. 9 control subjects and 36 individuals with FSHD were included in this study and patients were grouped based on expression levels of selected biomarkers. Groups 1 to 4 reflected higher DUX4 target gene expression and disease severity in an ascending order with the group 4 exhibiting the highest pathology scores [29]. Our analysis of the dataset (GSE115650) demonstrated that OSMR expression increased in all groups, with the highest levels observed in the group 4. OSMR was upregulated by 3.5-fold (Padj<0.000032) in all combined FSHD samples (FIG. 10F). 1-year follow-up assessment of muscle biopsies was published for the same patients (GSE140261), in which OSMR transcript levels remained high (n=27, fold change=3.1, Padj<0.00000022) (FIG. 10G) [30].

Finally, we also tested whether the OSM target genes were upregulated in muscular dystrophies. We performed GSEA analysis by using the top 200 genes upregulated by OSM. Our analysis determined that OSM target genes were significantly enriched in muscle biopsies of patients with DMD (GSE1007; normalized enrichment score (NES) =1.57, P-value<0.001) (FIG. 10H) and FSHD (GSE115650; NES=2.62, P<0.001 (FIG. 10I) and GSE140261; NES=2.6, P<0.001 (FIG. 10J)). Increased levels of OSMR transcript and the enrichment of OSM target genes in muscular dystrophies implicate the activation of the OSM/OSMR pathway in these diseases.

### Example 14. NIK Inhibition Blocks EDA-A2 and Tweak-induced Muscle Atrophy

The role of NIK activity in muscle atrophy was further studied utilizing a specific NIK small molecule inhibitor (NIK-SMI1 or SMI1 in short). When administered to cultured mouse primary myotubes, this inhibitor blocked the activation of the non-canonical NFκB pathway by muscle atrophy-inducing factors EDA-A2 and TWEAK. Accordingly, the electrophoretic mobility shift in NIK band representing the post-translational modification and the activation of this protein by EDA-A2 and TWEAK was completely abrogated in SMI1-treated samples (FIGS. 21A-21B), simultaneous NIK activation and inhibition resulted in massive accumulation of the non-modified protein in myotube cells (FIG. 21A and FIG. 21B). NIK inhibition by SMI1 reduced EDA-A2 and TWEAK-induced processing of p100-NFκB2 into p52-NFκB2, indicating the suppression of the downstream of non-canonical NFκB signaling (FIG. 21A and FIG. 21B). Atrophy of the primary myotubes detected by a reduction in the diameter of these cells was also inhibited by SMI1 when administered along with EDA-A2 and TWEAK (FIG. 21C and FIG. 21D). These results demonstrate that NIK activity is responsible for the induction of muscle atrophy by EDA-A2 and TWEAK. The results indicated that EDA2R-NIK signaling drives muscle wasting linked to cancer cachexia. Therefore, inhibition of NIK can be used to prevent muscle wasting.

### REFERENCES

1 Baracos, V.E., Martin, L., Korc, M., Guttridge, D.C. & Fearon, K.C. H. Cancer-associated cachexia. Nat Rev Dis Primers 4, 17105, doi:10.1038/nrdp.2017.105 (2018).
2 Cohen, S., Nathan, J.A. & Goldberg, A.L. Muscle wasting in disease: molecular mechanisms and promising therapies. Nature reviews. Drug discovery 14, 58-74, doi:10.1038/nrd4467 (2015).
3 Argiles, J.M., Stemmler, B., Lopez-Soriano, F.J. & Busquets, S. Inter-tissue communication in cancer cachexia. Nat Rev Endocrinol 15, 9-20, doi:10.1038/s41574-018-0123-0 (2018).
4 Dolly, A., Dumas, J.F. & Servais, S. Cancer cachexia and skeletal muscle atrophy in clinical studies: what do we really know? J Cachexia Sarcopenia Muscle 11, 1413-1428, doi:10.1002/jcsm.12633 (2020).
5 Kowalczyk-Quintas, C. & Schneider, P. Ectodysplasin A (EDA)—EDA receptor signalling and its pharmacological modulation. Cytokine Growth Factor Rev 25, 195-203, doi:10.1016/j. cytogfr.2014.01.004 (2014).
6 Yan, M. et al. Two-amino acid molecular switch in an epithelial morphogen that regulates binding to two distinct receptors. Science 290, 523-527 (2000).
7 Sadier, A., Viriot, L., Pantalacci, S. & Laudet, V. The ectodysplasin pathway: from diseases to adaptations. Trends Genet 30, 24-31, doi:10.1016/j.tig.2013.08.006 (2014).
8 Newton, K., French, D.M., Yan, M., Frantz, G.D. & Dixit, V.M. Myodegeneration in EDA-A2 transgenic mice is prevented by XEDAR deficiency. Mol Cell Biol 24, 1608-1613 (2004).
9 Das, S.K. et al. Adipose triglyceride lipase contributes to cancer-associated cachexia. Science 333, 233-238, doi:10.1126/science.1198973 (2011).
10 Kir, S. et al. Tumour-derived PTH-related protein triggers adipose tissue browning and cancer cachexia. Nature 513, 100-104, doi:10.1038/nature13528 (2014).
11 Sinha, S.K., Zachariah, S., Quinones, H.I., Shindo, M. & Chaudhary, P.M. Role of TRAF3 and -6 in the activation of the NF-kappa B and JNK pathways by X-linked ectodermal dysplasia receptor. The Journal of biological chemistry 277, 44953-44961, doi:10.1074/jbc.M207923200 (2002).
12 Verhelst, K. et al. XEDAR activates the non-canonical NF-kappaB pathway. Biochem Biophys Res Commun 465, 275-280, doi:10.1016/j.bbrc.2015.08.019 (2015).
13 Liu, T., Zhang, L., Joo, D. & Sun, S.C. NF-kappaB signaling in inflammation. Signal Transduct Target Ther 2, doi:10.1038/sigtrans.2017.23 (2017).
14 Awazawa, M. et al. A microRNA screen reveals that elevated hepatic ectodysplasin A expression contributes to obesity-induced insulin resistance in skeletal muscle. Nature medicine 23, 1466-1473, doi:10.1038/nm.4420 (2017).
15 Valino-Rivas, L. et al. NIK as a Druggable Mediator of Tissue Injury. Trends Mol Med 25, 341-360, doi:10.1016/j.molmed.2019.02.005 (2019).
16 Dogra, C. et al. TNF-related weak inducer of apoptosis (TWEAK) is a potent skeletal muscle-wasting cytokine. FASEB J 21, 1857-1869, doi:10.1096/fj.06-7537com (2007).
17 Johnston, A.J. et al. Targeting of Fn14 Prevents Cancer-Induced Cachexia and Prolongs Survival. Cell 162, 1365-1378, doi:10.1016/j.cell.2015.08.031 (2015).
18 Ren, X. et al. A small-molecule inhibitor of NF-kappaB-inducing kinase (NIK) protects liver from toxin-induced inflammation, oxidative stress, and injury. FASEB J 31, 711-718, doi:10.1096/fj.201600840R (2017).
19 Li, X. et al. Activation of NF-kappaB-Inducing Kinase in Islet beta Cells Causes beta Cell Failure and Diabetes. Mol Ther 28, 2430-2441, doi:10.1016/j.ymthe.2020.07.016 (2020).
20 Richards, C.D. The enigmatic cytokine oncostatin m and roles in disease. ISRN inflammation 2013, 512103, doi:10.1155/2013/512103 (2013).
21 Miki, Y. et al. Oncostatin M induces C2C12 myotube atrophy by modulating muscle differentiation and degradation. Biochem Biophys Res Commun 516, 951-956, doi:10.1016/j.bbrc.2019.06.143 (2019).
22. Richards, C.D., The enigmatic cytokine oncostatin m and roles in disease. ISRN Inflamm, 2013. 2013: p. 512103.
23. Bonetto, A., et al., JAK/STAT3 pathway inhibition blocks skeletal muscle wasting downstream of IL-6 and in experimental cancer cachexia. Am J Physiol Endocrinol Metab, 2012. 303(3): p. E410-21.
24. Cai, D., et al., IKKbeta/NF-kappaB activation causes severe muscle wasting in mice. Cell, 2004. 119(2): p. 285-98.
25. Silva, K.A., et al., Inhibition of Stat3 activation suppresses caspase-3 and the ubiquitin-proteasome system, leading to preservation of muscle mass in cancer cachexia. J Biol Chem, 2015. 290(17): p. 11177-87.
26. Judge, S.M., et al., Skeletal Muscle Fibrosis in Pancreatic Cancer Patients with Respect to Survival. JNCI Cancer Spectr, 2018. 2(3): p. pky043.
27. Dadgar, S., et al., Asynchronous remodeling is a driver of failed regeneration in Duchenne muscular dystrophy. J Cell Biol, 2014. 207(1): p. 139-58.
28. Haslett, J.N., et al., Gene expression profiling of Duchenne muscular dystrophy skeletal muscle. Neurogenetics, 2003. 4(4): p. 163-71.
29. Wang, L.H., et al., MRI-informed muscle biopsies correlate MRI with pathology and DUX4 target gene expression in FSHD. Hum Mol Genet, 2019. 28(3): p. 476-486.
30. Wong, C.J., et al., Longitudinal measures of RNA expression and disease activity in FSHD muscle biopsies. Hum Mol Genet, 2020. 29(6): p. 1030-1043.

What is claimed is:

1. An NFκB-inducing kinase (NIK) inhibitor for treating a skeletal muscle atrophy.

2. The NIK inhibitor according to claim 1, wherein the NIK inhibitor is used for treating the skeletal muscle atrophy linked to a cancer cachexia.

3. The NIK inhibitor according to claim 1, wherein the NIK inhibitor is used for treating a tumor-induced skeletal muscle atrophy.

4. The NIK inhibitor according to claim 1, wherein the NIK inhibitor is additionally used in combination with an Oncostatin M receptor (OSMR) inhibitor.

5. A method for treating a skeletal muscle atrophy in a subject, comprising administering a therapeutically effective amount of an NIK inhibitor to treat the skeletal muscle atrophy.

6. The method according to claim 5, wherein the NIK inhibitor is one or more of a small molecule, an antibody, an antisense oligonucleotide, a small interfering RNA (siRNA), or a short hairpin RNA (shRNA).

7. The method according to claim 5, wherein the NIK inhibitor is additionally used in combination with an OSMR inhibitor.

8. The method according to claim 5, wherein the subject is diagnosed with a tumor-induced skeletal muscle atrophy disease.

9. The method according to claim 5, wherein the subject is diagnosed with a skeletal muscle atrophy disease linked to a cancer cachexia.

10. A pharmaceutical formulation for treating a skeletal muscle atrophy, comprising a therapeutically effective amount of an NIK inhibitor as an active substance.

11. The pharmaceutical formulation according to claim 10, wherein the NIK inhibitor is one or more of a small molecule, an antibody, an antisense oligonucleotide, a siRNA, or a shRNA.

12. The pharmaceutical formulation according to claim 10, wherein the pharmaceutical formulation is administered by one of an oral route, a systemic route, a parenteral route, a nasal route, and an enteral route.

13. The pharmaceutical formulation according to claim 10, wherein the pharmaceutical formulation is indicated in a skeletal muscle atrophy disease, and the skeletal muscle atrophy disease is induced by a tumor or linked to a cancer cachexia.

14. A method for treating patients affected by a skeletal muscle atrophy disease, comprising the following steps:
a) selecting the patients affected by the skeletal muscle atrophy disease, and
b) administering a therapeutically effective amount of an NIK inhibitor alone or in combination with an OSMR inhibitor to the patients affected by the skeletal muscle atrophy disease.
